(19) United States
(12) Patent Application Publication
(10) Pub. No.: US 2019/0231745 A1
(43) Pub. Date: Aug. 1, 2019

(54) INHIBITORS OF T CELL RECEPTOR AND USES THEREOF

(71) Applicant: Consejo Superior De Investigaciones Cientificas, Madrid (ES)

(72) Inventor: Balbino Jose Alarcon Sanchez, Madrid (ES)

(21) Appl. No.: 16/225,587

(22) Filed: Dec. 19, 2018

Related U.S. Application Data
(60) Provisional application No. 62/607,943, filed on Dec. 20, 2017.

(51) Int. Cl. A61K 31/4025; A61P 17/06; A61P 11/06; A61P 25/28; A61P 37/02; A61K 9/00; A61K 31/352

(52) U.S. Cl. CPC A61K 31/4025; A61P 17/06; A61P 11/06; A61K 31/352; A61P 37/02; A61K 9/0053; A61P 25/28

(57) ABSTRACT

The present invention relates to selective inhibitors of TCR, and methods of using the same.

### TECHNICAL FIELD OF THE INVENTION

The present invention relates to methods useful for selective inhibition of T cell receptor (TCR). The content of Borroto et al., “First-in-class inhibitor of the T cell receptor for the treatment of autoimmune diseases”, Sci. Transl. Med. 2016, 8, 370ra184 is incorporated herein in its entirety by reference.

### BACKGROUND OF THE INVENTION

T lymphocytes play a central role in transplant rejection and, in a more or less direct way, in the generation of the autoimmune diseases. Therefore, current immunosuppressive drugs mechanisms of action are based on the inhibition of T lymphocyte activation. These immunosuppressants have highly toxic profiles, since they do not inhibit specific pathways for lymphocyte activation. T lymphocytes are activated through the antigen receptor (TCR) which recognizes the major histocompatibility complex (MHC) of the transplanted organ as foreign. The TCR is formed by six subunits, two of which (TCRα and TCRβ are responsible for the recognition of the MHC bound to antigen peptides while the other four (CD3γ, CD3δ, CD3ε and CD3ζ) are responsible for signal transduction to the lymphocyte cytoplasm (reviewed in Alarcon, B., Gil, D., Delgado, P. and Schamel, W. W. (2003) Immunol Rev, 191, 38-46). One of the initial processes that occur after binding of TCR by MHC is the activation of the tyrosine kinases of the src family, Lck and Fyn, which phosphorylate the tyrosines of the ITAM motifs of the CD3 subunits, which in turn become sites of anchorage of the tyrosines kinases of the Syk family (ZAP70 and Syk). Until recently it was thought that this was the linear scheme for signal transduction and that, from the kinases of the Sykfamily (ZAP70 mostly), a diverging activation cascade occurred resulting in the activation of various transcription factors, including NFAT, the target of the immunosuppressive drugs cyclosporine A and FK506 (Lin, J. and Weiss, A. (2001) J Cell Sci, 114, 243-244). Some years ago, the authors of the present invention discovered that, in order to be activated, the TCR undergoes a conformational change that results in the recruitment of the Nck adaptor directly to a proline-rich sequence (PRS) of the CD3ε subunit (Gil, D., Schemel, W. W., Montoya, M., Sanchez-Madrid, F. and Alarcon, B. (2002) Cell, 109, 901-912). This TCR-Nck interaction was shown to be essential for TCR activation by experiments involving the over-expression of the amino-terminal SH3.1 domain of Nck (which binds to CD3ε) and by the introduction of the APA1/1 antibody in T lymphocytes, which binds to PRS and blocks it. On the other hand, it has recently been described that Nck is necessary for T lymphocyte activation in response to stimulation of the TCR (Roy, E., Togbe, D., Holdorf, A. D., Trubetskoy, D., Nabti, D., Kiiblbeck, G., Klevenz, A., Kopp-Schneider, A. D., Leithauser, F., Moller, P., Bladt, F., Hammerling, G., Arnold, B., Pawson, T., and Tarufi, A. (2010) Proc Natl Acad Sci USA, 107, 15529-15534).

### SUMMARY OF THE INVENTION

Modulating T cell activation is critical for treating autoimmune diseases but requires avoiding concomitant opportunistic infections. Antigen binding to the T cell receptor (TCR) triggers the recruitment of the cytosolic adaptor protein Nck to a proline-rich sequence in the cytoplasmic tail of the TCR'sCD3ε subunit. Through virtual screening and using combinatorial chemistry, an orally available, low-molecular weight inhibitor of the TCR-Nck interaction was prepared that selectively inhibits TCR-triggered T cell activation with an IC50 (median inhibitory concentration) ˜1 nM. By modulating TCR signaling, the inhibitor prevented the development of psoriasis and asthma and, furthermore, exerted a long-lasting therapeutic effect in a model of autoimmune encephalomyelitis. However, it did not prevent the generation of a protective memory response against a mouse pathogen, suggesting that the compound might not exert its effects through immunosuppression. These results suggest that inhibiting an immediate TCR signal has promise for treating a broad spectrum of human T cell-mediated autoimmune and inflammatory diseases.

In one aspect, the present invention provides a method for treating an autoimmune and inflammatory disease in a patient comprising administering to the patient a compound of Formula A

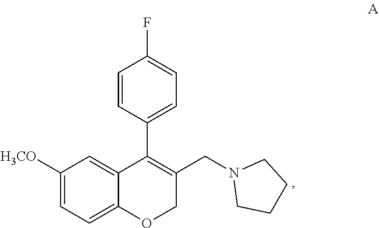 [SMILES: COc1ccc2c(c1)C(c1ccc(F)cc1)=C(CN1CCCC1)CO2]

or a pharmaceutically acceptable salt thereof.

### DESCRIPTION OF THE DRAWINGS

FIG. 1. Development of the lead compound AX-024. (A) The adaptor protein Nck is recruited to the PRS of CD3ε in the TCR. Antigen/MHC binding to the αβ subunits of the TCR induces the exposure of the PRS (blue box) in the cytoplasmic tail of CD3ε, enabling binding to the SH3.1 N-terminal domain of Nck. The three SH3 domains of Nck are represented as colored circles, and the C-terminal SH2 domain is represented as a yellow square. Immunoreceptor tyrosine-based activation motifs present in the CD3 cytoplasmic tails are represented as gray rectangles. (B) Surface model of the SH3.1 domain of Nck2 bound to the PRS of CD3ε, according to the Protein Data Bank (PDB) ID code 2JXB. The three hydrophobic pockets of SH3.1 that bind CD3ε are shown in green (pocket I) and marine blue (pocket II) for the PxxP motif, and in light cyan for the DY residues. Residues Trp⁴¹ and Lys³⁹ (amino acid position according to the PDB ID code 2JW4) of pocket DY are indicated. The CD3ε residues making up the PxxPxxDY SH3.1-binding motif are shown as sticks in magenta. (C) Effect of the top 10 inhibitors from the virtual screening on human blood T cell proliferation induced by an anti-CD3 antibody. Data represent means±SEM of the relative inhibition of T cell proliferation from triplicate data sets. (D) Structure of the AX-000 hit and AX-024 lead compounds. (E) Surface model of AX024 binding to pocket DY of Nck1(SH3.1) based on the PDB ID code 2JW4. AX-024 is shown as sticks, and the most important residues of SH3.1 conforming the pocket are indicated in a one-letter code. (F) Inhibition of human blood T cell proliferation by the AX-024 lead compound. Data represent means±SEM of the relative inhibition of T cell proliferation from triplicate data sets. (G) Histogram showing the averaged changes in ¹H and ¹⁵N chemical shifts for the peptide backbone of the labeled Nck1(SH3.1) protein produced on titration with AX-024 as a function of residue number [Δδ_{1H}=δ_{1H} [protein/AX024]−δ_{1H} [protein], parts per million (ppm), and Δδ_{15N} [protein/AX-024]−δ_{15N} [protein],ppm]. The line drawn at a Δδ^{av} of 0.010 indicates the level below which shifts are not significant. The positions of the amino acids displaying the most significant changes are indicated in a one-letter code. (H) Selected region of the 2D ¹H, ¹⁵N HSQC spectra showing some of the cross peaks of the Nck1(SH3.1) protein shifted upon titration with AX-024. The cross peaks corresponding to the peptide backbone and side chains (sc) of the indicated amino acids in the absence of AX-024 are shown with a black contour, and the cross peaks in the presence of AX-024 are shown with a red contour.

FIG. 2. Target specificity of AX-024. (A) The effect of AX-024 on B cell proliferation induced by stimulation of the BCR (anti-IgM), CD40, or TLR4 (LPS) was assessed on CellTrace Violet-labeled murine naïve spleen B cells after incubation for 72 hours. Proliferation was calculated according to CellTrace Violet dilution,and all data are compared to stimulated cells in the absence of compound for each stimulus. Data represent the relative proliferation (percentage±SEM of triplicate data sets) referred to as the stimulated samples in the absence of drug as 100%. The experiment was repeated three times. (B) The effect of AX-024 and AX-000 on IL-2-dependent T cell proliferation was measured in carboxyfluorescein diacetate succinimidyl ester (CFSE)-labeled human T cell lymphoblasts after incubation for 72 hours with IL-2 (10 ng/ml). A control of cell proliferation in the absence of IL-2 (unstimulated) is indicated by the blue square in the y axis. Data represent means±SEM of the T cell proliferation index generated from triplicate data sets. (C) The effect of AX-024 and AX-000 on T cell proliferation induced by PMA+ionomycin stimulation was measured by [3 H]thymidine incorporation into human blood T cells after 72 hours. A control of cell proliferation in the absence of stimulus is indicated by a blue square in the y axis. Data represent means±SEM of triplicate data sets. (D) The effect of AX-024 on antigen (Ag)-dependent T cell proliferation was tested by dilution of CellTrace Violet-labeled OT1 T cells from TCR transgenic WT and PRS knock-in mice (KI-PRS) after 5 days of stimulation with the OVA peptide derivative SIIQ-FEHL (Q4H7). Data represent means±SEM of the T cell proliferation index and CD25 mean fluorescence intensity (MFI) generated from triplicate data sets. (E) The effect of AX-024 on TCR binding to the Nck(SH3.1) and to the SH3 domain of Eps8L1, as well as on c-Cb1 binding to the Nck(SH3.1) domain, was assessed in pull-down (Pd) assays with GST fusion proteins. Postnuclear lysates of TCR-triggered (+) or unstimulated (.) Jurkat T cells were incubated with the indicated GST fusion proteins indicated in the presence of different concentrations of AX-024. Western blots (WB) were probed with the indicated specific antibodies and reprobed with anti-GST as a loading control. Plots show means±SEM of the relative expression of CD3z referred to as the loading control (GST), as quantified by densitometric scanning of three experiments. (F) Inhibition of CD3ε binding to Nck1(SH3.1) by AX-024 was evaluated by SPR using GST-Nck(SH3.1) recombinant protein for the solid phase and a synthetic peptide containing the PRS sequence of CD3ε in solution. Peptide association and dissociation rates were calculated in the continued presence of the concentrations of AX-024 indicated. RUs, response units. (G) Inhibition of anti-CD3-induced recruitment of endogenous Nck to the TCR by AX-024 was assessed by immunoprecipitation (Ip) with anti-CD3 and in Western blots probed with anti-Nck. Immunoblotting with CD3z was used as a loading control. The plot to the right shows means±SEM of the relative expression of Nck referred to as the loading control (CD3z), quantified by densitometric scanning of three experiments.

FIG. 3. Alteration of TCR signaling by AX-024. (A) The effect of AX-024 on TCR-triggered actin polymerization was measured by intracellular phalloidin staining of human blood T cells stimulated for 1 min with anti-CD3. A representative overlay of three histograms is shown (shadowed, nonstimulated cells; blue, stimulated without drug; red, stimulated with 1 nM AX-024). Quantitative data are represented below as the MFI (means±SEM of triplicate data sets) as a function of AX-024 concentration. The MFI value for nonstimulated cells is indicated with a blue square on the y axis. (B) Inhibition of ZAP70 phosphorylation (pZAP70) on Tyr³¹⁹ was examined by intracellular staining with a specific antibody in human blood T cells stimulated with anti-CD3 for 5 min. A representative overlay of three histograms is shown [as in (A)]. Quantitative data as in (A). The MFI value for nonstimulated cells is indicated with an open blue square on the y axis. (C) The effect of AX-024 on CD3ζ tyrosine phosphorylation was tested on postnuclear lysates of Jurkat cells stimulated for 5 min with anti-CD3 (+) or nonstimulated (−). Western blots were probed with anti-phosphorylated CD3ζ(pCD3ζ)Tyr¹⁴² and subsequently reprobed with anti-CD3ζ as a loading control. SDS-polyacrylamide gel electrophoresis was run under nonreducing conditions, and thereby, the two phosphorylated bands correspond to the phosphorylated forms of the CD3ζ homodimer. The plot to the right shows means±SEM of the relative expression of phosphorylated CD3z referred to as the loading control (CD3ζ), as quantified by densitometric scanning of three experiments. (D) Differential gene expression in purified blood T cells from two different human donors stimulated for 4 hours with anti-CD3 in the presence or in the absence of 1 nM AX-024. Gene expression differences between drug-treated and untreated samples from donor 1 are plotted in the x axis, whereas those found in samples from donor 2 are plotted in the y axis. Data are represented as the log₂ of the R fold ratio in untreated versus drug treated samples. R fold data were generated from triplicate data sets for each donor. The positions of data points referring to relevant cytokines and cytokine receptors inhibited by AX-024 are indicated in red. (E) Prediction of altered canonical signaling pathways deduced from mRNA expression data, according to the Ingenuity Pathway Analysis software. The table shows the P values of data referring to each canonical pathway and the genes altered in the presence of AX-024. Pathways referring to cytokine signaling are highlighted in blue, and pathways referring to TH cell differentiation or costimulatory receptor signaling are in red. ICOS, inducible T cell costimulator; ICOSL, ICOS ligand; JAK, Janus kinase; STAT, signal transducer and activator of transcription. (F) Prediction of upstream regulators affected by AX-024 according to mRNA expression and the Ingenuity Pathway Analysis, together with the P values and altered gene expression. Predicted alterations of TCR-CD3 and the costimulatory receptor CD28 signaling are highlighted in red, TGM2, transglutaminase 2; MAPK, mitogen-activated protein kinase.

FIG. 4. Anti-inflammatory effect of AX-024 in the IMQ model of psoriasis. (A) Reduction of scales and skin thickness in BALB/c mice topically treated with IMQ cream and single daily oral administrations of AX-024.HCl (10 mg/kg) for 5 days. Representative photographs of the shaved skin on mice's backs are shown, and the clinical scoring (means±SEM; n=10) was performed as indicated in Materials and Methods. P values were calculated by Student's t test. n.s., not significant. (B) Skin thickening, keratinocyte proliferation, and inflammatory cell infiltration in IMQ-treated mice evaluated by H&E and immunostaining with proliferation marker (Ki67)-specific and granulocyte marker (Ly6G)-specific antibodies. Arrowheads show the presence of haemorrhagic and infiltrated areas in H&E staining, the presence of granulocytes by Ly6G immunostaining, and the presence of proliferating cells in the basal layer of the epidermis (Ki67 immunostaining). The presence of granulocyte infiltrated epidermal scales in the vehicle group is indicated with an arrow. (C)Skin thickening was evaluated in random sections of the skin of IMQ-treated mice or control animals. Each dot represents a single measurement for a total of n=10 mice per group. Bars represent means±SEM of epidermis and dermis thickness. P values were calculated by Student's t test. (D) Infiltration of IMQ-treated ear skin by inflammatory cells was revealed by flow cytometry using common myeloid (CD11b), granulocyte (Ly6G), and CD8 T cell markers. Data represent means±SEM for n=5 mice per group. P values were calculated by Student's t test. (E) Presence of inflammatory cytokines in the skin on mice's backs, detected by qRT-PCR (means±SEM; n=5 mice per group). P values were calculated by Student's t test.

FIG. 5. Prevention of allergic airway disease by AX-024. (A) Sensitization and challenge in a model of OVA-induced allergic asthma. AX-024.HCl [50 mg/kg, intraperitoneally (ip) in saline] was administered twice at the indicated days. Mice were challenged with an aerosol of OVA on day 12 after the first immunization, and the recruitment of inflammatory cells to the lungs of the immunized mice was first examined in live animals 24 hours after administration of the aerosol by quantitative FMT and 48 hours after administration of the aerosol by cell population analysis in the BALF. (B) AX-024 inhibits infiltration of airways by inflammatory cells in OVA-sensitized mice, as measured by FMT using the ProSense 680 probe 24 hours after exposure to an OVA aerosol. A colorimetric scale was used to quantify the degree of infiltration. The ProSense signal was quantified and is shown on the right as means±SEM (n=4 mice per group). (C) The presence of inflammatory cells in BALFs was determined by flow cytometry using Gr-1 and CD11b as cell surface markers. Data represent means±SEM (n=4): *P<0.05 and **P<0.005 (Student's t test). (D) The presence of cytokines in BALF supernatants was determined by a multiplex assay, and the data represent means±SEM (n=4): *P<0.05 (Student's t test).

FIG. 6. Long-lasting prophylactic and therapeutic effects of AX-024 in the EAE model. (A) Effect of oral administration of daily doses of AX-024.HCl (10 mg/kg) for the first 10 days on subsequent development of neurological symptoms and weight loss. Data represent means±SEM (n=10, for both groups) where the P values were calculated by Student's t test. (B) The effect of AX-024 on CNS infiltration was assessed by immunostaining cryopreserved sections of the cerebellum and spinal cord of all mice sacrificed in (A). Inflammatory cell infiltration was calculated according to the number of CD4+ T cells and F4/80+ macrophages both in vessels and extravasated to the parenchyma (data represent means±SEM). P values were calculated by Student's t test. DAPI, 4′,6-diamidino-2-phenylindole. (C) The therapeutic effect of AX-024 was assessed in MOG-immunized mice by administering daily oral doses (10 mg/kg) of AX-024.HCl, starting on day 13 after immunization and ending on day 26 (temporal window of drug treatment indicated in the plot). Another group of mice received daily oral administrations of fingolimod (0.6 mg/kg) following the same schedule. Data represent means±SEM (n=10) of score and weight measurements. P values were calculated using the Kruskal-Wallis nonparametric test (red, AX-024 versus vehicle; blue, fingolimod versus vehicle). n.s., P>0.05.

FIG. 7. Effector cell differentiation in the presence of AX-024. (A) The effect of AX-024 on the differentiation of naive human CD4+ T cells to effector cells was studied by stimulation with anti-CD3 and anti-CD28 in the presence of the indicated concentrations of AX-024 in polarizing media favoring differentiation toward T_{H}1, T_{H}2, T_{H}17, or T_{regs}. Cells in culture were dual-stained with intracellular cytokines and FOXP3 and analyzed by flow cytometry. The dose-response effect of AX-024 on CD4 T cell differentiation is represented below as means±SEM of triplicate measurements. P values were calculated by Student's t test: *P<0.05, **P<0.005, and ***P<0.0005. (B) The inhibitory effect of CD4 T cells differentiated under T_{reg}-polarizing conditions (in the presence or absence of 1 nM AX-024) or under nonpolarizing conditions (T_{H}0) on proliferation of naive human CD4 T cells was measured after 5 days of stimulation with plate-bound anti-CD3. A 4:1 naïve T cells/Tregsratio was used. Data represent means±SEM of triplicate measurements. P values were calculated by Student's t test: **P<0.005 and ***P<0.0005. (C) Detection of FOXP3⁻, IFN-γ+, and IL-17A⁺ CD4⁺ T cells by flow cytometry in the draining popliteal lymph nodes of mice 6 days after immunization with MOG. Mice received daily oral doses of 10 mg/kg for 6 days or received the vehicle alone (vehicle). Data represent means±SEM (n=6, for both groups), where the P values were calculated by Student's t test. *P<0.05 and **P<0.005. (D) Cytokine release to the culture supernatant of T_{H}1 cells differentiated in the presence of the indicated concentrations of AX-024 as in (A) and stimulated for 24 hours with anti-CD3 or PMA+ionomycin in the absence of drug. Data represent means±SEM of triplicate measurements. P values were calculated by Student's t test: *P<0.0.

FIG. 8. Effector and memory immune response to a mouse pathogen in the presence of AX-024. (A) Evaluation of the activation of a CD8 T cell response to the immunodominant B8R poxvirus antigen peptide was carried out in mice immunized with autologous dendritic cells (DCs) loaded with B8R and treated with daily doses of oral AX-024 for seven consecutive days. The presence of B8R-reactive CD8 T cells was evaluated ex vivo after stimulation of spleen cells with the indicated doses of antigen peptide and intracellular staining with IFN-γ. Data represent means±SEM (n=3 mice per group). (B) First challenge: C57BL/6 mice were infected intranasally with the indicated doses of infective ECTV (in PFU) and treated with the indicated daily doses of AX-024.HCl orally (or vehicle alone, vehicle) for up to 10 days. Survival is individually represented for each mouse (n=5 mice per group). Rechallenge: Mice surviving the first infection were reinfected 60 days later with a lethal intranasal dose (10⁵ PFU) of ECTV. A control group of naíve mice, not exposed previously to ECTV, was infected in parallel. (C) CD8 T cell response to the immunodominant poxvirus antigen B8R was assessed in lymph node T cells of naïve and infected mice in (B) after stimulation with bone marrow-derived DCs loaded with B8R peptide. IFN-γ-producing cells were evaluated by intracellular staining on gated CD44⁺ CD69⁺ CD8 T cells. Data represent means±SEM (n=7 to 11 mice per group). (D) Diagram indicating affinity values and ligand potency for the OVA peptide derivatives OVAp (SIINFEKL), Q4R7 (SIIQ-FERL), and Q4H7 (SIIQFEHL), according to Daniels et al. (45). (E) Sensitivity to AX-024 inhibition in function of TCR affinity for the antigen ligand was tested by dilution of CellTrace Violet-labeled OT1 T cells from TCR transgenic WT mice after 4 days of stimulation with the indicated OVA peptide derivatives. Data represent means±SEM of the T cell proliferation index generated from triplicate data sets.

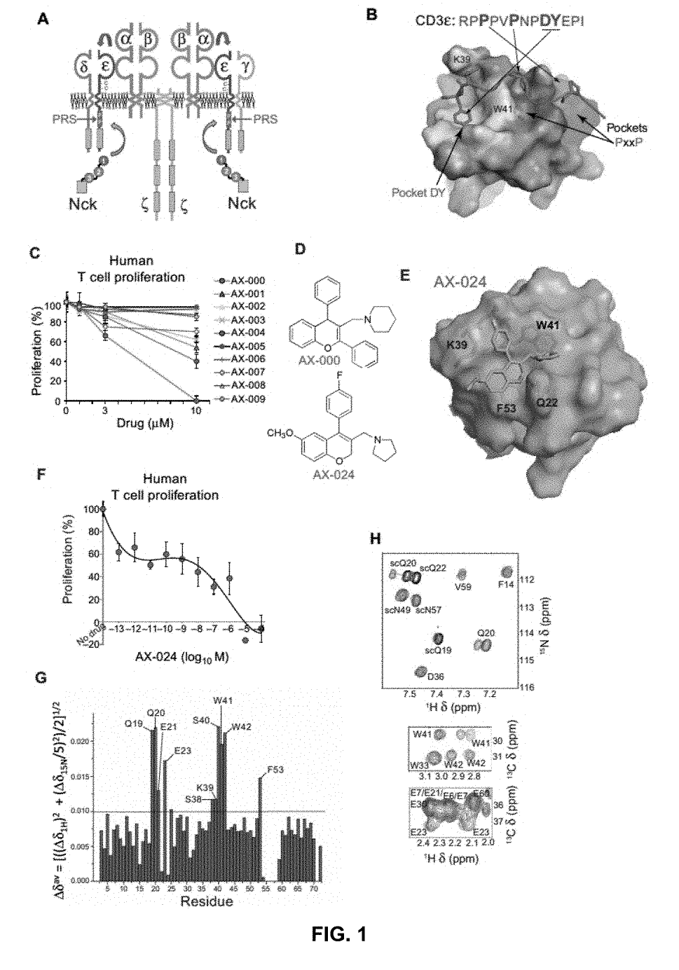

FIG. S1. Structure-activity relationship-based hit-to-lead process in the generation of the AX-024 lead compound. (A) Structures of the AX-000 hit and key intermediates in the hit-tolead process and surface models of binding to pocket DY of Nck1(SH3.1) based on PDB ID code 2JW4. Compounds are shown as sticks and the most important residues of SH3.1 conforming the pocket are indicated in a one-letter code. (B) Inhibition of human blood T cell proliferation in response to stimulation with plate-bound anti-CD3. Data represent the mean ±s.e.m. of the relative T cell proliferation from triplicate datasets. (C) Ball and stick model of AX-024 fitting in the DY cavity. The pose of AX-024 is the same as shown in FIG. 1E.

FIG. S2. Effect of hit and lead compounds on TCR-triggered cytokine release. The effect of AX-024 and the parental hit compound AX-000 on cytokine release was evaluated in the culture supernatant of human PBMC after 24 h stimulation with anti-CD3 and anti-CD28. Data represent the mean±s.e.m. of triplicate datasets.

FIG. S3. Comparison of the 2D 1H, 15N HSQC spectra of the SH3.1 domain of Nck1 shifted upon titration with AX-024 or with a CD3ε peptide. Sequence of the SH3.1 construct used for NMR experiments (top). Cloning-tag residues are shown in italics. Superposition of 2D ¹H, ¹⁵N-HSQC spectra of the SH3.1 domain of Nck1 at 150 μM concentration in the absence (black contours) and in the presence of AX-024 (red contours) at a protein/ligand ratio of 1:30. Cross-peaks in non-crowded regions are labelled. The two cross-peaks observed for the amide groups of Q and N side-chains (sc) are linked by a horizontal line. The cross-peaks for the NεH of Arg side-chains are indicated by an asterisk (*). (Bottom) Superposition of 2D ¹H, ¹⁵N-HSQC spectra of the SH3.1 domain of Nck1 at 20.7 μM concentration in the absence (black contours) and in the presence of peptide CD3ε (150-166) of sequence QRGQNKERPPPVPNPDY(green contours) at a protein/peptide ratio of 1:20. Cross-peaks in non-crowded regions are labelled. For some residues, cross-peaks for the free protein and for the protein/CD3ε (150-166) complex are linked by a line. The two cross-peaks observed for the amide groups of Q and N side-chains (sc) are linked by a horizontal line. The cross-peaks for the NεH of Arg side-chains are indicated by an asterisk (*). These data were taken from those reported in Ref (27).

FIG. S4. Validation of the target specificity of AX-024. (A) Overlay of amino acids conforming the DY pocket of Nck1(SH3.1) (magenta; PDB ID code 2JW4) with the corresponding amino acid residues in the DY pocket of the SH3 domain of Eps8 (grey; PDB ID code 1IOC). (B) The effect of AX-024 on TCR binding to the SH3 domain of Eps8 was assessed in a pull-down assay with GST fusion protein. Postnuclear lysates of TCR-triggered (+) or unstimulated (−) Jurkat T cells were incubated with 0.5 μg of GST-Eps8(SH3) fusion protein in the presence of different concentrations of AX-024. (C) Effect of AX-024 on Pak1 binding to the Nck(SH3.2) and on WASP binding to the Nck(SH3.3) was assessed in pull-down assays with GST fusion proteins. Western blots were probed with anti-Pak1 or anti-WASP and reprobed with anti-GST as a loading control. Plots show the mean±s.e.m. of the relative expression of Pak1 and WASP referred to the loading control, as quantified by densitometric scanning of 3 experiments.

FIG. S5. Effect of AX-024 on an actin-dependent process in non-T cells. Effect of AX-024 on human fibroblast cell migration in vitro was evaluated after producing a scratch with a P200 pipette tip and incubating for 24 hours in the presence of the indicated concentration of AX-024, cytochalasin D (1 μM) or vehicle alone. The yellow lines represent the central positions of the scratches.

FIG. S6. Effect of AX-024 on transcription of a selected set of genes in primary human T cells. Effect of different doses of AX-024 on mRNA expression of the indicated genes was assessed by RT-qPCR on samples of purified human blood T cells stimulated or not for 4 hours with plate-bound anti-CD3. Expression of the indicated genes was normalized to the expression of β-actin as a housekeeping gene and the values were divided by those of gene expression in non-stimulated cells in the absence of compound (set as a value of 1). Data represent the mean±s.e.m. of triplicate datasets.

FIG. S7. Toxicity of AX-024 in mice and rats. (A) Effect of distinct single doses of AX024. HCl administered intraperitoneally on body weight gain of CD-1 mice over 14 days. The data represent the mean±s.e.m. (n=10 per group). (B) Effect of distinct single doses of AX024. HCl administered orally on body weight gain of Swiss albino mice over 14 days. The data represent the mean±s.e.m. (n=2 per group). (C) Effect of distinct single doses of AX-024. HCl administered per orally on body weight gain of Sprague Dawley rats over 14 days. The data represent the mean±s.e.m. (n=2 per group). (D) Summary of acute toxicological findings in different species and calculation of the human equivalent dose.

FIG. S8. Effect of AX-024 on thymocyte differentiation. (A) Flow cytometry analysis of CD24 and TCR expression in thymocytes of 4 week-old C57BL/6 mice exposed to daily doses of oral AX-024 (10 mg/Kg and 40 mg/Kg) or to vehicle alone for 10 consecutive days. Both markers allowed the thymocytes to be divided into five subpopulations (a to e) that were reanalyzed according to CD4 and CD8 expression (right). The data below indicates the percentages of double negative (DN, CD4⁻ CD8⁻), double positive (DP, CD4⁺ CD8⁺) and single positive (CDSP, CD4⁺ CD8⁻; CD8SP. CD4⁻ CD8⁺) thymocytes within each a-to-e subpopulation. The data represent the mean±s.e.m. (n=6 mice per group). (B) Absolute cell number in the major thymic DN, DP, CD4SP, CD8SP subpopulations of mice analyzed in A. Numbers of pre- and post-beta selection (DN3 and DN4) and γδT cells are also indicated

FIG. S9. Anti-inflammatory effect of AX-024 in IMQ model of psoriasis. (A) Keratinocyte proliferation and inflammatory cell infiltration in imiquimod-treated mice were evaluated in random sections of hematoxilin and eosin (H&E) and immunostained skin. Plots represent the number of Ki67⁻ keratinocytes counted per section as a function of the length of the basal layer, and the percentage of subepidermic skin area infiltrated by Ly6G⁺ granulocytes as a function of the total subepidermic area. The data are represented as mean±s.e.m.; n=10 mice per group. (B) Presence of chemokines and chemoattractants in the skin on mice's backs detected by RTqPCR (mean±s.e.m.; n=10 mice per group).

FIG. S10. Inhibition of weight loss and neurological score in MOG-immunized mice by AX024. The AX-024 group was treated with a single daily dose of AX-024. HCl (50 mg/Kg, i.p.) for the first 10 days, while the Vehicle group received the vehicle alone. Two other groups received cladribine (1 mg/Kg mg. i.p.) or glatiramer acetate (5 mg/Kg, i.p.) with the same scheme as AX-024. Neurological scores were determined as described in the Methods section. Data represent the mean±s.e.m. (n=5, all groups except placebo or n=10, placebo group) where p values were calculated by Student's t-test.

FIG. S11. Primary SH3 domain sequence comparison. Comparison of SH3 domains was performed on the human Swiss-Prot database using BLAST search with the human Nck1(SH3.1) domain as the query sequence. Amino acids are represented in single-letter code. Bold format is used to highlight residues involved in formation of pockets I and II (for the PxxP motif), and the DY pocket. Amino acids conforming pockets I and II are in green; those conforming pocket DY are in red, blue and orange. Red indicates identity to the Nck1(SH3.1) pocket DY amino acids, orange similarity and blue disparity.

### DETAILED DESCRIPTION OF CERTAIN EMBODIMENTS

### 1. General Description of Certain Embodiments of the Invention:

Without wishing to be bound by any particular theory, it is believed that oral administration of the compound of Formula A, or a pharmaceutical acceptable salt thereof, results in selective inhibition of TCR triggered T cell activation, and thus can treat certain diseases, such as autoimmune and inflammatory diseases as described herein.

As used herein, the term “inhibitor” is defined as a compound that binds to and/or inhibits TCR with measurable affinity. In certain embodiments, an inhibitor has an IC50 and/or binding constant of less than about 100 μM, less than about 50 μM, less than about 1 μM, less than about 500 nM, less than about 100 nM, less than about 10 nM, or less than about 1 nM.

In one aspect, the present invention provides a method for treating an autoimmune and inflammatory disease in a patient comprising administering to the patient a compound of Formula A:

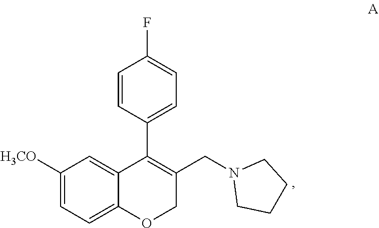 [SMILES: COc1ccc2c(c1)C(c1ccc(F)cc1)=C(CN1CCCC1)CO2]

or a pharmaceutically acceptable salt thereof.

### 2. Description of Exemplary Embodiments:

In some embodiments, the present invention provides a method for treating an autoimmune and inflammatory disease in a patient comprising administering to the patient a compound of Formula A:

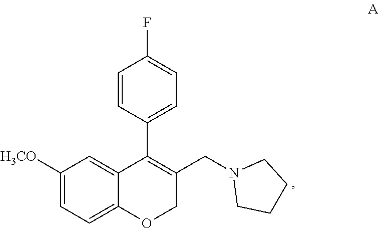 [SMILES: COc1ccc2c(c1)C(c1ccc(F)cc1)=C(CN1CCCC1)CO2]

or a pharmaceutically acceptable salt thereof.

In some embodiments, the present invention provides a method for selectively inhibiting TCR-Nck interaction in a patient comprising administering to the patient a compound of Formula A, as described herein, or a pharmaceutically acceptable salt thereof.

In some embodiments, the present invention provides a method for selectively inhibiting TCR-triggered T cell activation in a patient comprising administering to the patient a compound of Formula A, as described herein, or a pharmaceutically acceptable salt thereof.

In some embodiments, the present invention provides a method for specifically inhibiting the earlies TCR signaling events in a patient comprising administering to the patient a compound of Formula A, as described herein, or a pharmaceutically acceptable salt there.

In some embodiments, the present invention provides a method for treating psoriasis in a patient comprising administering to the patient a compound of Formula A, as described herein, or a pharmaceutically acceptable salt thereof. In some embodiments, the present compound attenuates severity of skin inflammation.

In some embodiments, the present invention provides a method for treating asthma in a patient comprising administering to the patient a compound of Formula A, as described herein, or a pharmaceutically acceptable salt thereof. In some embodiments, the present compound attenuates severity of lung inflammation.

In some embodiments, the present invention provides a method for treating multiple sclerosis in a patient comprising administering to the patient a compound of Formula A, as described herein, or a pharmaceutically acceptable salt thereof. In some embodiments, the present compound attenuates severity of neurological symptoms in the patient.

In some embodiments, the present invention provides a method for inhibiting effector T_{H} cell differentiation toward proinflammatory subsets in a patient comprising administering to the patient a compound of Formula A, as described herein, or a pharmaceutically acceptable salt thereof.

In some embodiments, the present invention provides a method for treating a T-cell mediated autoimmune and inflammatory disease in a patient comprising administering to the patient a compound that inhibits an immediate TCR signal. In some embodiments, the present invention provides a method for promoting T_{reg} differentiation in a patient comprising administering to the patient a compound that inhibits an immediate TCR signal. In some embodiments, the compound that inhibits an immediate TCR signal is a compound of Formula A, as described herein, or a pharmaceutically acceptable salt thereof.

In some embodiments, the present compound preserves T cell allevaion in response to pathogen-derived antigens.

In some embodiments, the present compound does not inhibit on T cell proliferation triggered by IL-2 or PMA+ionomycin.

In some embodiments, the present compound exerts a therapeutic effect that lasts after the drug is no longer detectable.

In some embodiments, the present compound is administered orally.

### 3. Uses and Administration:

The major histocompatibility complex (MHC) haplotype is the most significant genetic risk factor for human autoimmune diseases (ADs), thus drawing attention to T cells as major players of most immunopathological events. T cells recognize antigen peptides associated to MHC (pMHC) through T cell antigen receptors (TCR), comprising a complex of two antigen recognition subunits (TCRα and TCRβ) along with four signaling subunits (CD3γ, CD3δ, CD3ε, and CD3ζ). Several control mechanisms exist to avoid activation of T cells bearing TCRs with high affinity for MHC loaded with self-peptides, including deletion of potentially autoreactive T cells during their maturation in the thymus. However, these mechanisms are overridden in AD patients, and selfreactive T cells become activated and expand. Potentially autoreactive T cells are unable to cause disease when they emerge from the thymus because they must be activated by professional antigen presenting cells (APCs) to differentiate into harmful effector T cells. T cells require three signals for this activation to take place: signal 1, derived from the TCR; signal 2, derived from costimulatory receptors, for example, CD28 upon binding to its ligands on APCs; and signal 3, derived from cytokine receptors responsible for T cell proliferation and differentiation. Although the ultimate goal of therapeutic intervention in ADs is to stimulate immunological tolerance, the currently used agents seem more immunosuppressive than tolerogenic. Methotrexate, mycophenolate, azathioprine, and cladribine are cytostatic drugs, whereas antibodies like alemtuzumab (anti-CD52) induce T cell depletion. Furthermore, despite the central importance of the TCR signal for T cell activation in ADs, most current efforts to restrain T cell activation concentrate on modulating the second and third signals mentioned above. The anti-CD3 antibody OKT3 has been used successfully to treat acute rejection after allogeneic organ transplantation. However, this antibody induced severe adverse effects, such as cytokine release syndrome. This phenomenon is due to the fact that OKT3 is not a TCR-blocking antibody but rather an agonistic one triggering the TCR. This illustrates the priority regarding safety for chronic diseases such as ADs, which requires fine-tuning of T cell activation to prevent autoimmune attacks without suppressing immune responses against infectious agents. Therefore, the development of immunomodulators, preferably small drugs, which can interfere with the TCR signal, is an issue that has yet to be adequately addressed.

The TCR translates small differences in the chemical composition of the pMHC into quantitatively and qualitatively distinct outcomes, although the mechanism underlying this process remains poorly understood. Upon triggering, the TCR becomes phosphorylated by Lck at several tyrosine residues in the cytoplasmic tails of the CD3 subunits. This phosphorylation generates docking sites for the Syk family tyrosine kinase ZAP70. However, ZAP70 is not the only direct effector of the TCR, and recruitment of other proteins has also been described, including that of Nck, RRas2, Grk2 (G protein-coupled receptor kinase 2), and PI3K (phosphatidylinositol 3-kinase).

Nck is an adaptor protein that contains three tandem Src homology 3 (SH3) domains (SH3.1, SH3.2, and SH3.3) and a C-terminal SH2 domain. There are two highly similar Nck genes (Nck1 and Nck2) that are apparently redundant in terms of cell function. Nck universally coordinates signaling networks critical for actin cytoskeleton organization, cell movement, or axon guidance, connecting transmembrane receptors to multiple intracellular signaling pathways. Studies in double knockout mice lacking Nck1 in all tissues and conditionally lacking Nck2 in T cells set forth Nck as an important player in mature T cell function. In T cells, TCR triggering is followed by direct recruitment of Nck via its N-terminal SH3 (SH3.1) domain to a proline-rich sequence (PRS) in the cytoplasmic tail of CD3ε. More recently, we have demonstrated in a genetic complementation test that Nck recruitment to the PRS appears functionally downstream of the conformational change that takes place in the TCR upon pMHC binding. Experiments with Nck knockout mice and with mice bearing a mutated PRS indicate that the PRS-Nck interaction is important to activate mature T cells by weak but not strong agonists.

A new immunotherapy strategy for Ads is developed by modulating TCR activation using a new small chemical inhibitor of the Nck-CD3ε interaction. This inhibitor prevents full T cell activation in vitro with a median inhibitory concentration (IC50) below 1 nM, precluding or mitigating symptoms in animal models of AD and inflammatory diseases after oral administration, while sparing the assembly of a protective response to a viral pathogen after vaccination.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “TCR-mediated” disorders, diseases, and/or conditions as used herein means any disease or other deleterious condition in which TCR is known to play a role. Accordingly, another embodiment of the present invention relates to treating or lessening the severity of one or more diseases in which TCR is known to play a role.

In some embodiments, the present invention provides a method for treating one or more autoimmune and inflammatory diseases.

Autoimmune and Inflammatory Disorders and Conditions

In some embodiments, the present invention provides a method for treating autoimmune and inflammatory disorders and conditions in a patient comprising administering to said patient a compound of the present invention, or a pharmaceutically acceptable salt thereof, or a composition comprising said compound. Autoimmune and inflammatory disorders and conditions include, in one embodiment, without limitation, acute disseminated encephalomyelitis (ADEM), acute necrotizing hemorrhagic leukoencephalitis, Addison's disease, agammaglobulinemia, allergic asthma, allergic rhinitis, alopecia greata, amyloidosis, ankylosing spondylitis, anti-GBM/anti-TBM nephritis, antiphospholipid syndrome (APS), autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Behcet's disease, bullous pemphigoid, cardiomyopathy, Castleman disease, celiac sprue (nontropical), Chagas disease, autoimmune conditions associated with chronic fatigue syndrome or fibromyalgia, chronic inflammatory demyelinating polyneuropathy (CIDP), chronic recurrent multifocal ostomyelitis (CRMO), Churg-Strauss syndrome, icatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibrosing alveolitis, giant cell arteritis (temporal arteritis), glomerulonephritis, Goodpasture's syndrome, Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (see Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), sympathetic ophthalmia, Takayasu's arteritis, temporal arteritis/Giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, and Wegener's granulomatosis.

In some embodiments, the present invention provides a method for treating rejection of cell, organ and tissue graft transplants, graft versus host disease and/or for inducing xenograft tolerance, e.g., islet xenograft tolerance in a patient, comprising administering to said patient a compound of the present invention, or a composition comprising said compound.

In some embodiments, autoimmune and inflammatory disorders and conditions are associated with or causal to transplant rejection, including without limitation: a) Acute organ or tissue transplant rejection, e.g., treatment of recipients of, e.g., heart, lung, combined heart-lung, liver, kidney, pancreatic, skin, bowel, or corneal transplants, especially prevention and/or treatment of T-cell mediated rejection, as well as graft-versus-host disease, such as following bone marrow transplantation; b) Chronic rejection of a transplanted organ, in particular, prevention of graft vessel disease, e.g., characterized by stenosis of the arteries of the graft as a result of intima thickening due to smooth muscle cell proliferation and associated effects; c) Xenograft rejection, including the acute, hyperacute or chronic rejection of an organ occurring when the organ donor is of a different species from the recipient, most especially rejection mediated by B-cells or antibody-mediated rejection; d) Autoimmune disease and inflammatory conditions, in particular inflammatory conditions with an etiology including an immunological or autoimmune component such as arthritis (for example rheumatoid arthritis, arthritis chronica progrediente and arthritis deformans) and other rheumatic diseases.

In some embodiments, autoimmune and inflammatory disorders and conditions are autoimmune hematological disorders (including e.g., hemolytic anemia, aplastic anemia, pure red cell anemia and idiopathic thrombocytopenia), systemic lupus erythematosus, polychondritis, sclerodoma, Wegener granulomatosis, dermatomyositis, chronic active hepatitis, myasthenia gravis, psoriasis, Steven-Johnson syndrome, idiopathic sprue, (autoimmune) inflammatory bowel disease (including e.g. ulcerative colitis and Crohn's disease), endocrine ophthalmopathy, Graves' disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis, glomerulonephritis (with and without nephrotic syndrome, e.g., including idiopathic nephrotic syndrome or minimal change nephropathy) and juvenile dermatomyositis.

In some embodiments, autoimmune and inflammatory disorders and conditions are psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphigus, epidermolysis bullosa acquisita, and other inflammatory or allergic conditions of the skin, as are inflammatory conditions of the lungs and airways including asthma, allergies, and pneumoconiosis.

### Exemplification

The following examples are intended to illustrate the invention and are not to be construed as being limitations thereon.

### General Methods

### NMR Spectroscopy

NMR experiments were performed on either a Bruker AV-600 MHz or a Bruker AV-800 Mz spectrometer, both equipped with z-gradient cryoprobes. 2D ¹H-¹⁵N-HSQC and 2D ¹H-¹³C-HSQC spectra were acquired with standard Bruker pulse sequences, and sodium 2,2-dimethyl-2-silapentane-5-sulphonate (DSS) was used as an internal 1H chemical shift reference. ¹⁵N and ¹³C chemical shifts were indirectly referenced by multiplying the 1H spectrometer frequency assigned to 0 ppm by 0.251449530 and 0.101329118, respectively (Markley et al., 1998). NMR spectra were processed using TOPSPIN software (Bruker Biospin, Karlsruhe, Germany) and analyzed with Sparky (T. D. Goddard and D. G. Kneller, Sparky 3, University of California, San Francisco, USA).

To study ligand/protein interactions by NMR, a ¹³C, ¹⁵N-SH3.1 sample (˜150 μM; 0.5 mg in 400 ul H₂O/D₂O 9:1 v/v at pH 5.7) was titrated with increasing amounts of a stock solution of AX-024.HCl (˜5.8 mM in H₂O/D₂O 9:1 v/v at pH 5.7). 2D ¹H-¹⁵N-HSQC spectra and 2D ¹H-¹³C-HSQC spectra were recorded at 25° C. for the free protein and for each titration point.

### Surface Plasmon Resonance

HBS-EP was used as the running buffer (10 mM HEPES [pH 7.4], 150 mM NaCl, 3 mM EDTA, 0.005% [v/v] Surfactant P20). Immobilization of the GST-Nck1(SH3.1) purified recombinant protein (final concentration 30 μg/ml) was performed by the amino coupling strategy using sodium acetate (10 mM) at pH 4.5 and 150 μL of a solution of EDC/NHS (v/v), with optimum immobilization achieved at approximately 5000 resonance units. For interaction experiments, increasing concentrations of the CD3ε peptide RGQNKERPPPVPNPDY and AX-024 were used on the same chip. In all cases, the flow rate was 10 μl/s. After each interaction, 2.5 M glycine (pH 2) was used to regenerate the surface, and the analyte response after regeneration was constant after repeated injections in all the experiments and within ±10% of the response following the first injection. The raw SPR data were prepared for global analysis by BIA Evaluation 3.2 (General Electric), and the corrected binding data were then analyzed by directly fitting the curve to a simple biomolecular interaction mechanism (1:1) and mass transport effect. These were fit to the association and dissociation phase sensor data in all the experiments, and the error space for each of the parameters was assessed using statistical profiling (Rmax<0.001).

Differentiation of Human Th Cells in vitro

The cytokine and antibody cocktails used during the first three days were: Th1: IL-12 (25 ng/ml) and anti-IL-4 (5 ng/ml). Th2: IL-4 (10 ng/ml) and anti-IFNγ(5 ng/ml). Th17: TGFβ(5 ng/ml), IL-6 (100 ng/ml), IL-1β(10 ng/ml), anti-IL-4 (5 ng/ml) and anti-IFNγ(5 ng/ml). Treg: TGFβ(2 ng/ml) and IL-2 (5 ng/ml). Three days after stimulation, Th1, Th2 and Treg cells were washed and cultured in the absence of CD3 and CD28 stimulation for 2 additional days in the following conditions: Th1: IL-12 (12 ng/ml), IL-2 (4 ng/ml), and anti-IL-4 (3 ng/ml). Th2: IL-4 (5 ng/ml), IL-2 (4 ng/ml), and anti-IFNγ(3 ng/ml). Treg: IL-2 (2 ng/ml) and TGFβ(2 ng/ml).

### Cytokine Release

Fresh human T cells enriched from whole blood by density centrifugation. Myeloid and B cells were removed by negative selection with a cocktail of biotinylated antibodies against CD11b, B220 and Gr1, and streptavidin-coated magnetic beads (Dynal). The remaining population contained over 94% T cells, which were then stimulated for 24 h on 96-well plates coated with OKT3 (10 μg/ml) and soluble anti-CD28 (1 μg/ml) in the presence of inhibitors. The supernatants were collected and the secreted cytokines analyzed using a BD CBA human cytokine kit.

### Immunoblot Analysis of T Cell Activation

Cells were lysed in 1 ml of Brij96 lysis buffer containing protease and phosphatase inhibitors (0.3% Brij96, 140 mM NaCl, 20 mM Tris-HCl [pH 7.8], 10 mM iodoacetamide, 1 mM PMSF, 1 μg/ml leupeptin, 1 μg/ml aprotinin, 1 mM sodium orthovanadate and 20 mM sodium fluoride). After removing the nuclei by low speed centrifugation, cell lysates were resolved by SDS-PAGE and immunoblotted using standard protocols. The membranes were probed with anti-CD3ζ serum 448(62), anti-phospho ERK or anti-total ERK antibodies (Cell Signaling), visualizing the protein bands by ECL (Pierce).

For Jurkat T cell stimulation, a total of 3×107 cells were incubated for different times with an equal number of Raji APCs preloaded with Staphylococcus enterotoxin E “superantigen” (0.1 mg). Cells were lysed in 1 ml of Brij96 lysis buffer, and immunoprecipitation from the postnuclear supernatants was performed with anti-CD3 OKT3 and protein A Sepharose beads. After SDS-PAGE and immunoblotting, the membranes were probed with an anti-Nck antibody (Exbio) and reprobed with anti-CD3ζ serum.

For co-immunoprecipitation analysis of endogenous Nck, a total of 4×107 enriched human blood T-cells were pre-incubated in serum-free RPMI medium with compound AX-024 before stimulation with soluble OKT3 (10 μg/ml) for 1 minute at 37° C. Cells were lysed as above, and immunoprecipitation was performed with OKT3 and protein A Sepharose beads. The resulting immunoprecipitated extracts were resolved in a 10% acrylamide SDS-PAGE gel for immunoblotting with a rabbit anti-Nck antibody and reprobed with anti-CD3ζ serum.

### Phosphoflow Analysis of Zap70 Phosphorylation

A total of 1×106 PBLs were incubated at 37° C. in the presence of the compound AX-024 at the indicated concentrations in serum-free RPMI medium for 1 hour prior to stimulation with soluble OKT3 anti-CD3 antibody (10 μg/ml) for 5 minutes. Subsequently, cells were fixed in 2% paraformaldehyde, permeabilized with 0.1% NP40 and incubated with rabbit anti-phospho ZAP70 (Y319) (Cell Signaling) followed by an anti-rabbit Ig Alexa fluor 647 antibody (Invitrogen). Phosphorylation levels of ZAP70 were quantified by flow cytometry.

### Microarray Analysis of Gene Expression

Total CD4⁺ and CD8⁻ T-cells were isolated by negative selection from PBLs and resuspended at a density of 5×106 cells/ml in serum-free medium and stimulated in the presence of different concentrations of AX-024 on culture plates coated with anti-CD3 (clone OKT3) at 10 μg/ml for 4 hours. RNA was extracted using the RNAeasy kit according to the manufacturer's instructions (QIAGEN 74134) and its integrity was assessed using an Agilent 2100 Bioanalyzer (Agilent). Labelling and hybridization were performed according to the protocols from Affymetrix. Briefly, 100 ng of total RNA were amplified and labelled using the WT Plus reagent kit (Affymetrix) and then hybridized to Human Gene 2.0 ST Array (Affymetrix). Washing and scanning were performed using the Affymetrix GeneChip System (GeneChip Hybridization Oven 645, GeneChip Fluidics Station 450 and GeneChip Scanner 7G).

### EAE Severity Scale

0=normal behavior; no overt signs of disease; 1=weakness at the distal portion of the tail; 1.5=complete flaccidity of the tail; 2=moderate hind limb weakness; 2.5=severe hind limb weakness; 3 ataxia; 3.5=partial hind limb paralysis; 4=complete hind limb paralysis; 4.532 complete hind limb paralysis accompanied by muscle stiffness; 5=Moribund state and hence sacrifice for humane reasons.

### EAE Histological Evaluation

For histological evaluation of CNS infiltration, the cerebellum and the spinal cord were washed in phosphate buffer saline (PBS), cryo-protected overnight in 30% sucrose/PBS solution, embedded and frozen in a 7.5:15% gelatin/sucrose solution and serial-sectioned in the sagittal (cerebellum) or transversal (spinal cord) plane at 15□m thickness, using a cryostat (Leica). Cryosections were permeabilized with PBS containing 0.1% Triton X-100 (PBT) and immunostained in PBT containing 1% normal goat serum with the following primary antibodies: rabbit anti-laminin (1:500, Sigma), CD4 and F4/80. Primary antibodies were detected with Alexa488- or Alexa568-conjugated secondary antibodies (Molecular Probes, Eugene, Oreg.). Sections were counterstained with DAPI (1 μg/ml, Vector).

### Imiquimod Model of Psoriasis.

Mice 8 to 11 weeks old received a daily topical dose of 50 mg of commercially available Imiquimod cream (5%) (Aldara; 3M Pharmaceuticals) on their shaved back and their right ear (half dose) for 5 consecutive days, translating into a daily dose of 2.5 mg in the skin on their back or 1.25 mg per ear of the active compound. Daily oral doses of AX-024.HCl (10 mg/Kg) in saline were administered by oral gavage for 5 days.

### Flow Cytometry Analysis of Psoriatic Skin

Ears were collected and dissected into dorsal and ventral halves. The samples were digested with Liberase TM (Roche) diluted at 0.25 mg/ml in serum-free RPMI medium for 60 min at 37° C. After the incubation period, the enzyme was inhibited by adding 50 ml of PBS supplemented with 0.05% of BSA and 0.05 mM of EDTA (PBS-BSA-EDTA) and mechanically disrupted by passing through a 70-micron cell strainer to obtain a skin cell suspension. Incubation of skin cell suspensions with anti-mouse FcRII/III (clone 2.4G2) for 10 min at 4° C. in PBS-BSA-EDTA solution was routinely carried out prior to staining. For flow cytometryanalysis, the following anti-mouse antibodies from BD Bioscience were used: CD45, CD11b, Ly6G and CD8.

### Scoring Severity of Skin Inflammation

To score inflammation severity on the skin from their backs, an objective scoring system was followed based on the clinical Psoriasis Area and Severity Index (PAST), as previously described (38). Erythema, scaling, and thickening were scored independently on a scale from 0 to 4: 0, none; 1, slight; 2, moderate; 3, marked; and 4, very marked. The level of erythema was scored using a scoring table with red taints. The cumulative score (erythema plus scaling plus thickening) served as a measure of the severity of inflammation (scale 0-12).

### Immunohistochemistry

Samples of skin from mice's backs were rapidly immersed in liquid nitrogen and stored at −80° C. until use for quantitative PCR, or fixed in 4% paraformaldehyde and embedded in paraffin. For the histological study, skin slices (4-5 μm thick) were stained with H&E and analyzed by two blinded evaluators. For IHC staining, skin sections were deparaffinized, boiled in antigen retrieval solution (10 mM sodium citrate, 0.05% Tween 20, pH6), and incubated with the primary rabbit monoclonal anti-mouse Ki67 (Master Diagnostica) and rat monoclonal anti-mouse Ly6G antibodies (Abcam), followed by specific secondary antibodies from Dako: envision flex system for Ki67 and rabbit anti-rat HRP for Ly6G. Slides were developed with DAB substrate (DakoK3468) and then counterstained with Mayer's Hematoxylin. Quantification of epidermal and dermal thickness as well as number of cells positive for Ki67 and Ly6G were calculated for several skin sections of at least 4 randomly selected mice per group.

### Analysis of Cytokine Expression in Mouse Skin Samples

Quantification of cytokine expression in mouse skin samples was performed on total RNA extracted from skin biopsies. Skin biopsy samples were homogenized in 2 ml of Trizol reagent in a Polytron PT 3000 probe for tissue disruption. RNA was purified by chloroform extraction and isopropanol precipitation. Reverse transcription was performed using the Superscript III first-strand synthesis system kit (Life Technologies). Subsequent quantification of gene expression was performed by qPCR using the GoTaq® qPCR Master Mix (Promega) in an ABI PRISM 7900HT SDS device (Life Technologies). The following oligonucleotides were used to quantify cytokine and chemokine expression:

TNFα:
5′CAGGCGGTGCCTATGTCTC 3′,
5′GATCACCCCGAAGTTCAGTAG 3′;
IL-1β:
5′GCAACTGTTCCTGAACTCAACT 3′,
5′′ATCTTTTGGGGTCCGTCAACT 3′;
CXCL2:
5′CCAACCACCAGGCTACAGG 3′,
5′GCGTCACACTCAAGCTCTG 3′;
CXCL9:
5′ GGAGTTCGAGGAACCCTAGTG 3′,
5′GGGATTTGTAGTGGATCGTGC 3′;
S100A9:
5′ GCACAGTTGGCAACCTTTATG 3′,
5′TGATTGTCCTGGTTTGTGTCC 3′;
S100A8:
5′AAATCACCATGCCCTCTACAAG 3′,
5′CCCACTTTTATCACCATCGCAA 3′

### In Vivo FMT 1500 Tomographic Imaging in the OVA-Induced Allergic Asthma Model

Mice were injected intravenously with 4 nmol of Prosense 680 probe (PerkinElmer, Inc.), and OVA-challenged and control mice were then analyzed using the FMT 1500 fluorescence tomography in vivo imaging system (PerkinElmer, Inc.). The fluorescence data collected were reconstructed by FMT 2500 system software True Quant version 3.0 for three-dimensional fluorescence quantification. The total amount of lung fluorescence (in picomols) was calculated relative to internal standards.

### Ectromelia Infection

Each group consisted of 5 animals, which were monitored every day until 21 days p.i. and then on days 28, 35 and 42. Mice were anesthetized with 0.1 ml/10 g body weight of ketamine HCl (9 mg/ml) and xylazine (1 mg/ml) by i.p. injections. Anesthetized mice were laid on their dorsal side with their bodies angled so that the anterior end was raised 45° from the surface; a plastic mouse holder was used to ensure conformity. ECTV was diluted in PBS without Ca²⁺ and Mg²⁺ to the required concentration and slowly loaded into each nare (5 μl/nare). Mice were subsequently left in situ for 2-3 min. before being returned to their cages. At the indicated times following exposure to ECTV, groups of mice were treated by oral gavage with 0.1 ml sterile, distilled water (vehicle) or water containing the desired concentration of AX-024. To determine infectious viral titres, mice were sacrificed post challenge, and lung, spleen, liver tissues and nasal-wash were isolated. Tissue was ground in PBS (10% w/v), frozen and thawed three times, and sonicated for 20 seconds. Virus infectivity (PFU/ml) in tissue homogenates was estimated by titration on BSC-1 monolayers. Arithmetic means were calculated for PFU/ml values above the limit of detection (1×10²PFU/ml). The remaining mice were observed for clinical signs of disease (morbidity) and mortality. Moribund mice were euthanized.

### EXAMPLE 1

### SYNTHESIS OF 2H-benzopyran DERIVATIVES

The general method used to prepare the title compounds is shown below. Briefly, the corresponding chroman-4-one derivative was allowed to react with the appropriate phenylmagnesium salt followed by dehydration in acid medium to give the 4-substituted benzopyran adduct 2. Formylation of this intermediate using standard conditions yielded aldehyde 3, which was finally converted into the desired compound 4 by reductive amination. The commercial availability of the starting chroman-4-one that bears an additional phenyl group at C-2 facilitated the preparation of compound AX-024. Purification of final compounds was carried out by chromatography (preparative thin-layer or column) on silica gel.

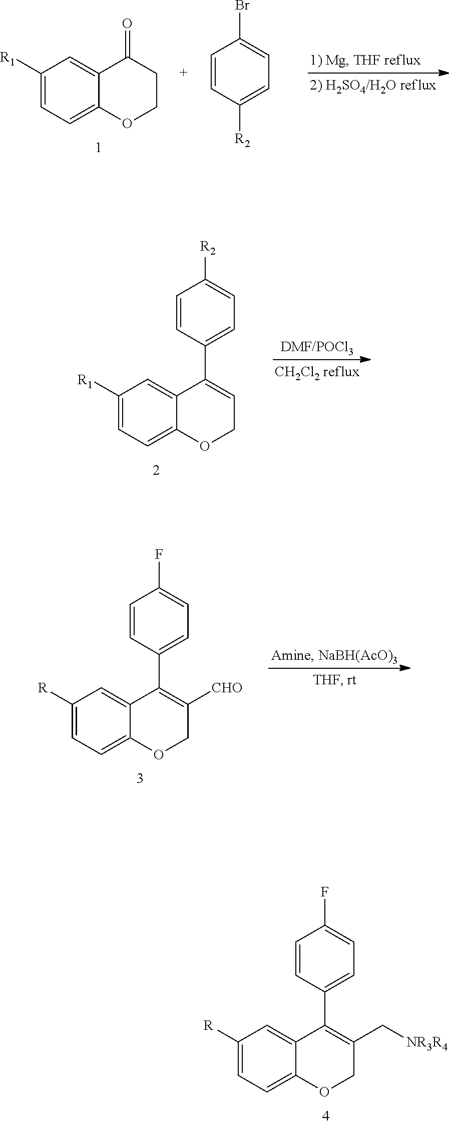

The synthetic sequence involved well-known reactions that give good overall yields of the final compounds, which were then purified by silica gel chromatography (preparative thin-layer or column). Rigorous structural characterization of the intermediates and compounds was carried out by analytical (HPLC) and spectroscopy (¹H and ¹³C NMR, and HRMS). For preparation of the hydrochloride salt, a solution of AX-024 base in methanol (1 mmol/ml, total volume of 46 ml) was cooled to 0° C. in an ice-water mix and hydrochloric acid (4.6 mmol in 0.93 ml) was added dropwise. The mixture was allowed to react for 1 hour and the solvent was eliminated at low pressure.

### EXAMPLE 2

### IN VITRO AND IN VIVO TESTING OF THE COMPOUNDS

The study was designed to investigate whether recruitment to the TCR of Nck through the PRS of CD3ε was a pharmacologically amenable target for the treatment of T cell-mediated diseases. The sample sizes chosen for the drug treatment were adequately designed to observe the effects on the basis of past experience and studies of this type conducted by others. Animals were only excluded in case of an accident during immunization or if drug administration resulted in severe damage. Mice and rats receiving vehicle or drugs were randomized after immunization. The identity of the animals that received vehicle or drugs was masked to researchers who scored them. The identity was revealed after the data had been collected. Details on sampling and experimental replicates are provided in each figure legend.

Virtual screening: Putative ligands for virtual screening were obtained from the publicly available ChemBridge database in SMILES format (51), and they were processed with our in-house VSDMIP platform according to our standard protocol (26, 52. Further information regarding chemical library preparation, receptor preparation, binding site characterization, virtual screening, and molecular modeling of AX-024 is described in the general methods described herein.

NMR spectroscopy: NMR experiments were performed as described in the general method described herein.

Surface plasmon resonance: SPR was performed on a Biacore X Instrument (General Electric) setup according to the manufacturer's instructions, using CM5 as a sensor chip. Immobilization and interaction details are provided in general methods described herein.

Cells and mice: Human blood samples were obtained from the Center for Blood Transfusions of the Comunidad de Madrid, where donations were obtained from healthy volunteers after providing their informed consent. Murine lymph node T cells were maintained in RPMI and 10% fetal bovine serum supplemented with 20 mM b-mercaptoethanol and 10 mM sodium pyruvate. All mice were maintained under specific pathogen-free conditions at the animal facilities of the Centro de Biologia Molecular Severo Ochoa, the Fundación Centro Nacional de Investigaciones Cardiovasculares Carlos III, or the Instituto de Investigaciones Biomédicas August Pi i Sunyer, in accordance with current national and European guidelines (Directive 2010/63/EU). All animal procedures were approved by the ethical committees of the three institutes.

T cell proliferation: Human T cell proliferation in response to anti-CD3 was measured by CFSE dye dilution. Fresh human peripheral blood lymphocytes (PBLs) were obtained by density centrifugation of whole blood, labeled with 4 μM CFSE and incubated for 4 days at 37° C. on anti-CD3 antibody OKT3-coated 96-well plates in the presence of inhibitors. Alternatively, fresh human PBLs were CFSE-labeled and stimulated for 4 days at 37° C. with a mixture of PMA (10 ng/ml) and 1 mM ionomycin in the presence of inhibitors. Cells were stained with APC-labeled anti-CD4 and analyzed in a FACSCalibur flow cytometer. The proliferation index was calculated according to the number of cell divisions and the percentage of cells in each CFSE peak of the CD4⁺ population. Human T cell lymphoblasts were generated from PBLs by stimulation with phytohemagglutinin (2 μg/ml) in RPMI with 10% fetal bovine serum. After 2 days, the cultures were washed and expanded in medium with IL-2 for 5 days. Human lymphoblasts were subsequently washed, labeled with CFSE, and stimulated with IL-2 (100 ng/ml) for 3 days further in the presence of inhibitors. Human T cell proliferation in response to PMA+ionomycin was analyzed by [³H]thymidine incorporation 48 hours after stimulation with PMA (10 ng/ml) and ionomycin (1 μM) in the presence of the indicated concentrations of AX-024. Lymph node T cells from OT1 TCR transgenic WT or PRS knock-in mice (KI-PRS) (28) were labeled with CellTrace Violet (Life Technologies), incubated for 1 hour in the presence of different concentrations of AX-024, and subsequently stimulated for 3 days with bone marrow-derived dendritic cells (DCs) preloaded with different peptide antigens at a ratio of 1.5×10⁵ T cells/3×10⁴ DCs. DCs were preloaded with either the agonist OVA peptide (SIINFEKL) (100 pM) or the OVA peptide variant Q4H7 (SIIQFEHL) (10 nM). At the time of analysis, cells were stained with anti-CD8-peridinin chlorophyll protein and anti-CD25-APC antibodies (BD Pharmingen). Cell proliferation was analyzed by CellTrace Violet dilution as described above for CFSE dilution.

B cell proliferation: Spleen B cells from C57BL/6 mice were labeled with CellTrace Violet and incubated for 72 hours with either anti-IgM (10 μg/ml) or anti-CD40 (5 μg/ml), supplemented with IL-4 (5 ng/ml) or LPS (2.5 μg/ml) in the presence of different concentrations of AX-024. Proliferation was calculated according to the total number of cell divisions (by Cell-Trace Violet dilution).

Differentiation of human TH cells in vitro: Naïve human blood CD4⁺ T cells were activated with plate-bound anti-CD3 (5 μg/ml), anti-CD28 (5 μg/ml), and different cytokine and antibody cocktails for 3 days, washed, and cultured for two additional days in the absence of CD3 and CD28 stimulation under conditions described in general methods as described herein. Five days after activation, the cells were washed and restimulated for 4 hours with PMA and ionomycin in the presence of GolgiStop. Intracellular expression of IL-17A, IFN-γ, IL-4, IL-2, and FOXP3 and extracellular expression of CD25 were analyzed after staining with specific antibodies.

Cytokine release: Fresh human T cells enriched from whole blood as described in the general methods herein were used to measure secreted cytokines using a BD CBA kit.

Immunoblot analysis of T cell activation: A total of 3×107 enriched human T cells were stimulated with soluble OKT3 anti-CD3 antibody (10 μg/ml) for different times or stimulated with Raji APC cells, lysed, and analyzed by Western blot, as described in the general methods herein.

Microarray analysis of gene expression: Microarray analysis of gene expression is described in the general methods herein.

Acute toxicity: Eight-week-old CD-1 mice were injected intraperitoneally with different amounts of the hydrochloride salt of AX-024 (AX-024.HCl) dissolved in 0.5 ml of saline. All animals were observed clinically for the appearance of macroscopically visible adverse reactions twice daily over 14 days, as well as immediately after AX-024 administration. A necropsy was carried out on each animal on day 14, and the abdominal, thoracic, and cranial cavities were examined in situ, together with their associated organs.

Experimental autoimmune encephalomyelitis: All these studies were approved by the Ethics and Scientific Committees of the University of Barcelona. Chronic EAE was induced in female C57BL/6 mice (6 to 8 weeks old; 20-g body weight) by subcutaneously injecting a total of 150 μg of MOG35-55 (Espikem) emulsified in Freund's complete adjuvant (Sigma-Aldrich) and supplemented with Mycobacterium tuberculosis (1 mg/ml) (H37Ra strain from Difco) into both femoral regions. The mice were immediately injected intraperitoneally with 200 ng of pertussis toxin (Sigma-Aldrich) and, again, 48 hours after immunization. The animals were weighed and inspected for clinical signs of disease on a daily basis by an observer blind to the treatments. Disease severity of EAE was assessed according to the scale described in the general methods herein. At the end of the study, the animals were anesthetized and perfused intracardially with 4% paraformaldehyde in 0.1 M phosphate buffer (pH 7.6). The brains and spinal cords of the mice were dissected out and fixed. Daily doses of AX-024 (0.01, 0.1, and 1 mg per mouse per dose), glatiramer acetate (0.1 mg per mouse per dose), and cladribine (0.02 mg per mouse per dose) were prepared in phosphate-buffered saline (PBS) and administered intraperitoneally. In the prevention trials, treatments were given for 10 days starting on the day of immunization, whereas in the therapeutic trial, the treatment began when more than 50% of the animals reached a score higher than 2 and was administered until the end of the experiment. Histological and immunohistochemical evaluations are described in the general methods herein.

IMQ model of psoriasis: BALB/c mice were purchased from Harlan and kept under specific pathogen-free conditions with food and water ad libitum. All experimental procedures were approved by the local animal ethics committee according to Spanish and European guidelines. The IMQ-induced model of psoriasis was conducted following the protocol described by van der Fits et al.(34) and in the general methods herein. Flow cytometry analysis of psoriatic skin was performed on mouse ears, and the scoring severity of mouse back skin inflammation was assessed as described in the general methods herein. Immunohistochemistry and cytokine expression analyses were carried out on mouse back skin following the protocols described in the general methods herein.

OVA-induced allergic asthma model: BALB/c mice (10 to 12 weeks old) were injected with OVA (15 mg, ip) (Sigma) in 200-μl alum (Pierce). The injection was repeated on days 5 and 12, and mice were challenged with aerosolized 0.5% OVA in PBS (two 60-min challenges administered 4 hours apart). Control animals were exposed to inhaled PBS alone. AX-024.HCl was injected intraperitoneally on days 0 and 5, 2 hours before OVA administration. Mice were sacrificed on day 14, 40 hours after the aerosol OVA challenge, and BALF was collected or analyzed in vivo by FMT imaging on day 13. When analyzing BALF, different populations of leukocytes were identified by their expression of CD11b, Gr-1, B220, and CD11c. IL-4, IL-5, and other effector T cell cytokines were measured in BALF supernatants with a FlowCytomix Mouse T_{H}1/T_{H}2 10 plex kit (Bender MedSystems GmbH), followed by flow cytometry in BD FACSCanto II Cytometer (BD Biosciences). In Vivo FMT 1500 Tomographic Imaging was performed as described in the general methods herein.

Ectromelia infection: The reference ectromelia strain used was ECTV-Naval.Cam (complete genome sequence available at www.poxvirus.org). ECTV was grown in BSC-1 cells. For infection of mice, virus stocks were purified by centrifugation through a 36% sucrose cushion. Four- to 6-week-old female C57BL/6 mice were obtained from Harlan laboratories, and housed in filter-top microisolator cages in an animal biosafety level 3 containment area. Animal husbandry and experimental procedures were in accordance with Public Health Service policy and approved by the Institutional Animal Care and Use Committee. Details regarding ECTV injection and AX-024 treatment are described in the general methods herein.

Statistical analyses: Data are reported as means±SEM of multiple individual experiments each carried out in triplicate. Unless stated otherwise, the statistical analysis was carried out with GraphPad Prism 6.0. A two-tailed t test was used if two groups were compared, and a nonparametric Kruskal-Wallis test was used when three groups were simultaneously compared. Differences were considered significant if P<0.05. For SPR data, statistical analysis was performed with MathLab v7.8 (2009) software, considering a normal distribution for each data set (kₒₙ and k_{off}) per feature and per assay, and using a median absolute deviation to gain a robust measure of the statistical dispersion of the data obtained. The values (kₒₙ and k_{off}) outside of the criteria (average±3 SD) were removed, and the affinity constant (KD) was established as k_{off}/kₒₙ values.

### Results

### 2.1. A Hit-To-Lead Process Results in a Potent Modulator of T Cell Activation

Nck is recruited through its SH3.1 domain to the PRS of CD3ε (FIG. 1A) (18). The PRS of CD3ε interacts with three shallow hydrophobic pockets in SH3.1 (21-23). In CD3ε, the canonical PxxP sequence involved in PRS-SH3 interactions is followed by two amino acids at position +3 that establish interactions with the third SH3.1 pocket (FIG. 1B, the DY pocket). Thus, the interaction sequence in the PRS of CD3ε would be more accurately represented by the motif PxxPxxDY. Whereas the pockets for the two central proline residues are conserved in most SH3 domains (24), the DY pocket is uncommon and has only been described in a handful of Eps8 family proteins (21, 25). Furthermore, this pocket is constituted by aromatic and polar amino acid residues in the SH3.1 domain of Nck1 and Nck2 (Q20, Q22, E23, K39, T40, W41, and Y53 in Nck2), which are not found in other SH3 domains (table S1). We hypothesized that a high-affinity low-molecular weight compound that binds to the DY pocket might act as a selective inhibitor of the Nck-CD3εinteraction. We set out to validate this hypothesis by means of a docking-based virtual screening approach performed on ligands from the ChemBridge database (www.chembridge.com/index.php) using our in-house platform (26). The best score for each ligand was selected as the docking solution. Ten of the highest-scoring compounds were selected and tested experimentally through in vitro proliferation assays on human peripheral blood T cells stimulated with anti-CD3. Two compounds showed inhibitory properties at concentrations below 10 mM (FIG. 1C), of which the most promising candidate was AX-000 (FIG. 1D), which was therefore selected as the hit for the hit-to-lead optimization process.

For optimization, we used the predicted fit of AX-000 in the DY pocket of SH3.1 (FIG. S1A). According to this model, the 2H-benzopyran moiety sits at the bottom of the shallow DY pocket, establishing p-stacking interactions with the lateral chains of aromatic amino acids W41 and F53. AX-000 optimization was carried out by removing and adding substituents to the 2H-benzopyran moiety to subsequently compare the resulting compounds in human T cell proliferation assays. Molecular dynamics simulations suggested that the AX-000 phenyl substituent in position 2 is exposed to the aqueous solvent. This substituent hindered compound activity, whereas the piperidine substituent in position 3 of 2H-benzopyran favored its action (compare AX-000 with AX-0D9, AX-0A3, and AX-004; FIG. S1,A and B). Replacing the piperidine substituent by a smaller pyrrolidine ring and adding a methoxy substituent in position 6 gave rise to the lead compound AX-024, which was >10,000-fold more potent than the AX-000 hit in terms of inhibition of TCR-triggered T cell proliferation (FIG. 1,D and F). The IC50 of AX-024 in this assay was 1 nM, although it showed inhibitory effects at a concentration of 1 pM or less (FIG. 1F). AX-024 was also a much more potent inhibitor of cytokine release by human peripheral blood mononuclear cells stimulated with anti-CD3 than AX-000, strongly hindering interleukin-6 (IL-6), tumor necrosis factor-a (TNFa), interferon-g (IFN-γ), IL-10, and IL-17A production at a concentration of 10 nM (FIG. S2).

The molecular dynamics simulation of predicted AX-024 docking suggests that AX-024 occupies most of the cavity (FIG. S1C). The cavity has a total volume of 718.4 Å³, of which 264.0 Å³ is occupied by AX-024, roughly 37%. In contrast, the compound covers all the solvent-accessible surface area (SASA) of the pocket [the pocket has a SASA of 245.0 Å², and AX-024 has a three-dimensional (3D) SASA of 522.1 Å²]. The interaction is prevalently through van der Waals' forces, except for a hydrogen bridge that is formed between the tertiary nitrogen of the pyrrolidine ring and the carbonyl group of the Q22 lateral chain (FIG. 1E). The fluorophenyl group in position 4 is oriented toward K39, establishing hydrophobic interactions with the hydrocarbon region of the K39 side chain and with W41. The hydrocarbon moiety of the pyrrolidine group interacts via van der Waals forces with W41 and the methoxy group in position 6 with the hydrophobic part of K39. The methoxy group also reinforces the p-stacking interactions of the 2H-benzopyran nucleus with F53.

To demonstrate that AX-024 did indeed bind to the DY pocket of the Nck SH3.1 domain, we performed nuclear magnetic resonance (NMR) ligand/protein interaction studies using a double ¹³C, ¹⁵N-labeled sample of SH3.1(Nck1). The AX-024 hydrochloride salt was prepared to increase the compound solubility in water and was added to the protein sample (50 mM) at increasing protein/ligand ratios. Some cross peaks in the 2D ¹H, ¹⁵N heteronuclear single-quantum coherence (HSQC) and ¹H, ¹³C HSQC spectra of the SH3.1(Nck1) protein shifted upon titration (FIG. 1,G and H; details of all amino acids are in FIG. S3), confirming that AX-024 binds to this Nck domain. Because the NMR spectra of the SH3.1(Nck1) domain had been previously assigned (22), we could identify which residues were affected by AX-024 binding and map the AX024 binding site. The most substantial changes observed corresponded to the ¹H, ¹⁵N cross peaks of the backbone amide groups of Q19, Q20, E21, E23, S38, K39, S40, W41, W42, and F53 (FIG. 1G); the side-chain amide groups of Q19, Q20, and Q22; and the ¹H, ¹³C cross peaks of the side-chain protons of Q22, E23, and W41 (two selected regions of the spectrum are shown in FIG. 1H). All these residues either contribute to the DY pocket (Q20, Q22, E23, S40, W41, and F53) or are very close to it (Q19, S38, and W42), and therefore, the NMR data confirmed that AX-024 bound to the DY pocket of the Nck SH3.1 domain. As expected, the residues showing shifts corresponded to only a fraction of those affected by binding of a CD3ε peptide encompassing the entire PRS (FIG. S3), because the peptide not only binds to the DY pocket but also to the two pockets for the PxxP motif (FIG. 1B and FIG. S3).

### 2.2. AX-024 Specifically Inhibits TCR-Dependent T Cell Activation

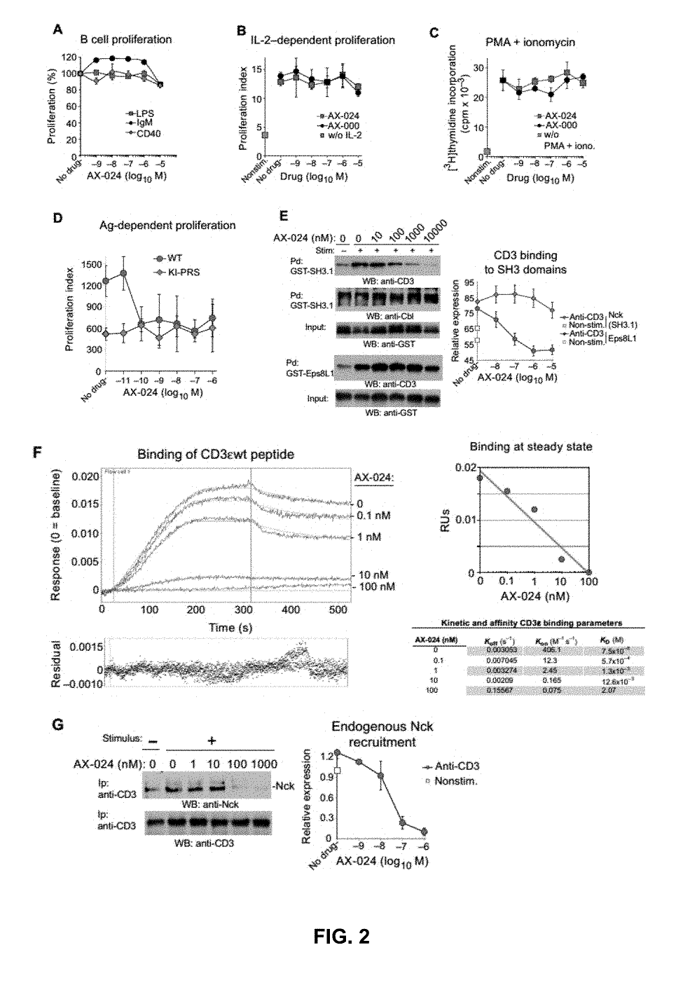

B cell antigen receptor (BCR) triggering also recruits Nck (27). However, and unlike described previously for T cells, Nck recruitment to the BCR is mediated by the SH2 domain of Nck and is independent of the Nck SH3.1 domain. Consequently, BCR-triggered B cell proliferation [anti-immunoglobulin M (IgM) stimulation] was not inhibited by AX-024 at concentrations as high as 10 μM (FIG. 2A). Likewise, B cell proliferation induced by the Toll-like receptor 4 (TLR4) and CD40 agonists LPS (lipopolysaccharide) and anti-CD40, respectively, was not inhibited. To determine whether the inhibitory effect of AX-024 on T cell activation is selective of stimuli that trigger the TCR, we evaluated its effects on the IL-2-dependent proliferation of T lymphoblasts, which is no longer dependent on TCR signaling. Neither AX-024 nor the parental compound AX-000 inhibited T lymphoblast proliferation at concentrations up to 10 μM (FIG. 2B), suggesting that they did not inhibit general housekeeping activities in T cells. T cell proliferation induced by a combination of phorbol 12-myristate 13-acetate (PMA) and ionomycin, agents that bypass TCR signaling by activating downstream signaling pathways, was unaffected by AX-024, suggesting that AX-024 targets a TCR-proximal event. In CD8⁺ T cells of OT1 TCR transgenic (OT1Tg) mice bearing wild-type (WT) AX-024 strongly inhibited T cell proliferation at a concentration of 0.1 nM when OT1Tg T cells were WT for the PRS mutation (FIG. 2D). In the absence of inhibitor, OT1Tg PRS knock-in mouse T cells with a germline mutation in the PRS of CD3ε (KI-PRS) that prevents Nck recruitment to the TCR (28, 29) proliferated nearly threefold less than WT OT1Tg cells. However, incubation with AX-024 did not exert further inhibitory effects on T cell proliferation than those caused by the PRS mutation (FIG. 2D), reflecting that AX-024 specifically targets the Nck-CD3ε interaction.

The selectivity of AX-024 for the Nck-CD3ε interaction is not surprising given that it targets an atypical pocket, that is, the DY pocket, which is only present in a few proteins of the Eps8 family, in addition to the SH3.1(Nck) domain (30). One member of this family, Eps8L1, has been described to bind the PRS of CD3ε (31). However, the conservation of amino acid residues that make up the DY pocket (shown in red in table S1) is low in Eps8 family members as well as in all SH3 domains (FIG. S4A and table S1). The PRS is only exposed for SH3.1(Nck) binding when the TCR is triggered adopting the “open” conformation (18). Thus, in a pull-down assay with an immobilized glutathione S-transferase (GST)-SH3.1(Nck) fusion protein, we noticed an increase in the abundance of TCR that is able to bind SH3.1(Nck) in lysates of T cells stimulated with anti-CD3 (FIG. 2E). To assess the effect on binding of the entire TCR, Western blotting was carried out with an anti-CD3z antibody rather than with an antibody against the PRS-containing CD3ε subunit, to avoid detection of nonassembled cytoplasmic CD3ε. The inducible binding of the TCR through its PRS was also found when a GST-SH3 (Eps8L1) recombinant protein was used in the pull-down assay (FIG. 2E), indicating that the inducible binding of SH3 domains to the PRS is a general property of the triggered TCR. However, although AX-024 inhibited TCR binding to SH3.1(Nck) with an IC50<0.1 mM in this assay, it did not affect binding to SH3(Eps8L1) (FIG. 2E) or SH3(Eps8) (FIG. S4B) even at a concentration of 10 mM. Furthermore, c-Cb1 binding to SH3.1(Nck), which is not dependent on the DY pocket, was not affected by AX-024 (FIG. 2E). As expected, interactions of other SH3 domains with polyproline sequences not mediated by DY pockets were not affected by AX-024 (FIG. S4C).

To more precisely estimate the IC50 of AX-024 inhibition of TCR binding to SH3.1(Nck), we carried out surface plasmon resonance (SPR) measurements of the binding kinetics of a synthetic peptide corresponding to the PRS of CD3ε to an immobilized SH3.1(Nck1) domain. We injected CD3εwt peptide alone or in combination with different concentrations of AX-024 (0.1 to 100 nM) and detected an inhibitory effect even at low concentrations (FIG. 2F). To estimate an IC50 for the inhibition of CD3ε peptide binding, we plotted the maximum steady-state response units at different AX-024 concentrations and found that it is about 1 nM (FIG. 2F). A kinetic assay was performed with different concentrations of the CD3ε peptide alone or in the presence of 0.1 to 100 nM concentrations of AX-024 to derive a KD of 7.5 mM for the peptide in the absence of drug and a 173-fold decrease in affinity in the presence of 1 nM AX-024 (FIG. 2F).

We also determined whether AX-024 altered the recruitment of endogenous Nck to CD3ε, and therefore to the TCR, in human blood T cells stimulated with anti-CD3. Co-immunoprecipitation experiments in these cells showed that Nck recruitment to the TCR was induced upon stimulation in the absence of drug but was inhibited in the presence of AX-024 in a dose-dependent manner at concentrations starting from 1 nM (FIG. 2G).

### 2.3. AX-024 Specifically Inhibits the Earliest TCR Signaling Events

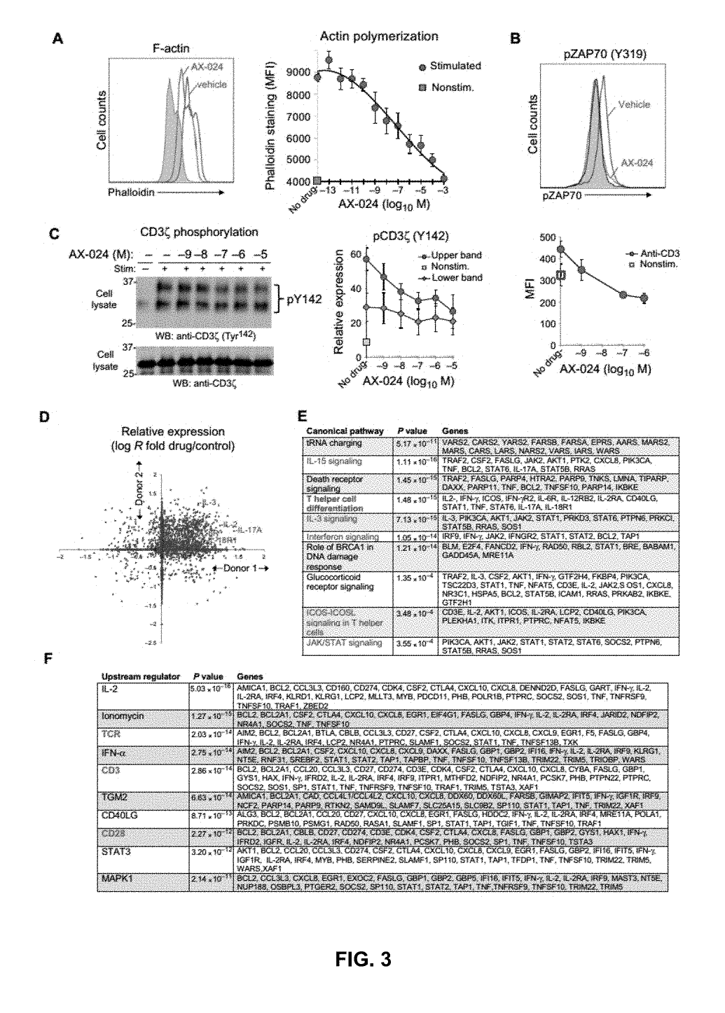

Because Nck is a critical regulator of the actin cytoskeleton, we assessed whether AX-024 impaired its remodeling upon TCR triggering. Stimulating human blood T cells with anti-CD3 elicited a 2.5-fold increase of intracellular F-actin, which could be inhibited by AX-024 with an IC50 of 1 nM (FIG. 3A). By contrast, AX-024 did not affect fibroblast migration in a wound-healing assay, which itself is a process that depends on actin cytoskeleton rearrangement (FIG. S5). In addition, AX-024 inhibited phosphorylation of ZAP70 at Tyr³¹⁹ (FIG. 3B) and of CD3ζ at Tyr¹⁴² (FIG. 3C), which represent two early hallmarks of TCR triggering. The inhibitory effect on tyrosine phosphorylation of CD3ζ was especially apparent for the low-mobility, fully phosphorylated form of the protein.

To explore the consequences regarding gene expression upon AX024 inhibition of the TCR-Nck interaction, we carried out whole genome microarray transcriptomic analysis of purified human blood T cells stimulated with anti-CD3 for 4 hours, and the experiment was repeated using T cells from two different blood donors. T cell stimulation in the presence of a concentration of AX-024 as low as 1 nM had an impact on gene transcription (FIG. 3D). Although AX-024 treatment up-regulated the expression of a few genes compared to stimulated control cells, the effect of AX-024 was mostly to inhibit TCR triggering-induced gene expression. Precisely 1093 genes were transcriptionally downregulated in the presence of AX-024 in a reproducible manner, including genes that are relevant for inflammatory T cell responses, for example, cytokines IL-17A, IL-2, and IFN-γ or cytokine receptors IL-6R and IL18R1 FIG. 3D). An analysis of the impact of AX-024 treatment on mRNA expression using the Ingenuity Pathway Analysis software indicated that AX-024 was altering canonical pathways typically activated by proinflammatory cytokines (IL-15, IL-3, and IFN-γ) and affecting transcription of genes involved in T helper (T_{H}) cell differentiation (FIG. 3E). Gene expression analysis also allowed predicting upstream regulators affected by AX-024, including cytokines and tissue transglutaminase 2, a central mediator of inflammation (FIG. 3F). The effect of AX-024 on gene expression also predicted that the drug was altering TCR (and CD3) signaling, thus reinforcing the biochemical and flow cytometry data in support of AX-024 inhibiting proximal TCR signaling. Furthermore, although we only used an anti-CD3 antibody for T cell stimulation in these experiments, that is, not including a CD28 ligand, the impact of AX-024 on gene expression pointed to an alteration of genes typically up-regulated by CD28 (FIG. 3F).

The effect of AX-024 on the expression of a selected set of genes was also analyzed by quantitative reverse transcriptase polymerase chain reaction (qRT-PCR) in human T cells stimulated with anti-CD3 for 4 hours. Whereas the TCR-induced expression of some genes (for example, CXCL9, CXCL10, and EGR3; FIG. S6) was abrogated at a concentration of 1 nM, the response of other genes was more gradual (for example, ZRANB2 and CD96) or even bimodal, with stronger inhibition at lower concentrations than at higher concentrations (for example, IL-2 and IFN-γ; FIG. S6). These results suggest that the effect of AX-024 on T cell activation is not a mere decrease in TCR signaling but rather results in a complex set of effects suggestive of immunomodulation.

### 2.4. AX-024 Attenuates the Severity of Skin Inflammation in a Psoriasis Model as Well as of Lung Inflammation in an Asthma Model

Before we tested the efficacy of AX-024 on animal models of ADs, we first tested acute toxicity in mice after a single intraperitoneal injection at four different doses (2.8, 14, 70, and 350 mg/kg). Mice followed for 14 days showed no significant adverse reactions to AX-024, and all mice gained weight at a similar rate (FIG. S7A). Toxicity after single dose oral administrations was also evaluated in mice and rats. Single doses as high as 400 mg/kg (mice) or 1600 mg/kg (rats) were administered without any adverse effects on body weight (FIG. S7,B and C), on organ size, or on the generation of lesions visible after necropsy (FIG. S7D). These results showed that AX-024 administration was not toxic for rodents after single administration of doses as high 1600 mg/kg. As for thymocyte differentiation, it is impaired yet not blocked in mice bearing the germline mutation in the PRS of CD3ε (KI-PRS) at every step in which pre-TCR or TCR signaling is required (29). However, the administration of AX-024 for 10 days to 4-week-old mice did not have a significant impact on thymocyte subset numbers, suggesting that AX-024 does not seriously affect thymic differentiation (FIG. S8).

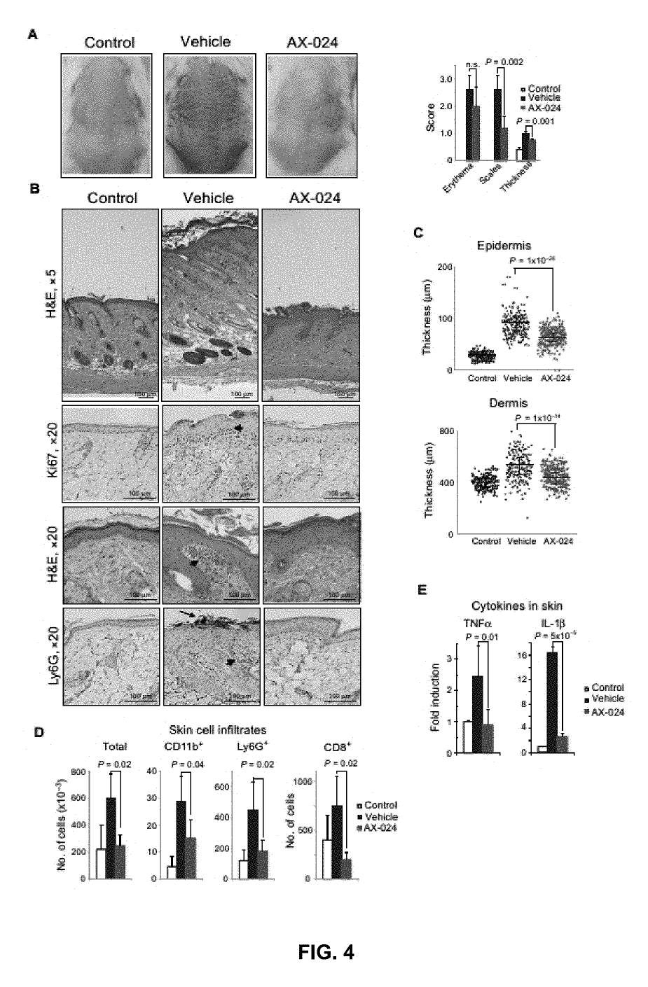

Psoriasis is a chronic inflammatory relapsing/remitting skin disease characterized by red, scaly, and often itchy patches, papules, and plaques that cover from small areas of the skin to the whole body (32). We tested the effect of AX-024 on the prevention of parakeratosis, epidermal hyperplasia, and cellular infiltration after administration of the TLR7 and TLR8 agonist imiquimod (IMQ). Upon topical administration, IMQ reproduces many human psoriasis symptoms as well as IL-17 and IL-23 axis dependence (33, 34). Mice received a daily topical dose of IMQ cream on their backs and their right ear for five consecutive days. A daily dose of AX-024.HCl (10 mg/kg) was administered orally for 5 days just before each IMQ administration, and the severity of inflammation of the back skin was scored on day 5. The AX-024-treated group presented less scales and reduced skin thickening compared to the vehicle group (FIG. 4A). A more quantitative assessment of skin thickening was obtained by hematoxylin and eosin (H&E) staining of back skin sections and by subsequently measuring epidermal and dermal thickness at multiple sites chosen randomly. Treatment with AX-024 significantly reduced thickening of both skin layers, but more effectively of the dermis, which rather resembled that of mice treated with a control cream lacking IMQ (FIG. 4,B and C). Epidermis thickening in the vehicle group correlated with an increase in the number of basal layer keratinocytes positive for the Ki67 marker of cells undergoing division (FIG. 4B, ×20 magnification, arrowhead). The number of Ki67+ keratinocytes in the AX-024-treated group was reduced (FIG. 4B), although this reduction was not statistically significant (FIG. S9A). Higher magnification of H&E-stained sections highlighted the presence of areas of hemorrhage and intense infiltration with mononuclear and polymorphonuclear cells under the epidermis in the vehicle but not in the other two groups (FIG. 4B, ×20 magnification, arrowhead). Furthermore, immunohistochemical staining with a Ly6G-specific antibody revealed numerous foci of infiltrating granulocytes that were localized subepidermally in the vehicle but not in the control or AX-024 groups [FIG. 4B (×20 magnification, arrowhead) and FIG. 4B). Skin samples derived from mice in the vehicle group also presented with profuse scales that were positive for Ly6G staining (FIG. 4B, arrow), whereas these were scanter in AX-024-treated mice (FIG. 4A, histogram). Flow cytometry analysis after collagenase treatment was used to further evaluate skin infiltration by inflammatory cells. Treatment with AX-024 inhibited skin infiltration by granulocytes (Ly6G+ cells), as well as by all CD11b+ myeloid cells and by CD8+ T lymphocytes (FIG. 4D). This reduced infiltration was accompanied by a strong reduction of proinflammatory cytokines (TNFa and IL-1b) and chemokines (CXCL2), as well as neutrophil chemoattractants (S100A8 and S100A9), as detected by qRT-PCR in skin samples (FIG. 4E and FIG. S9B). The cytokines IL17A and IL-22 were not detectable in any condition. Together, these results indicate that AX-024 is effective at attenuating the psoriasis-like symptoms of inflammation in the IMQ model.

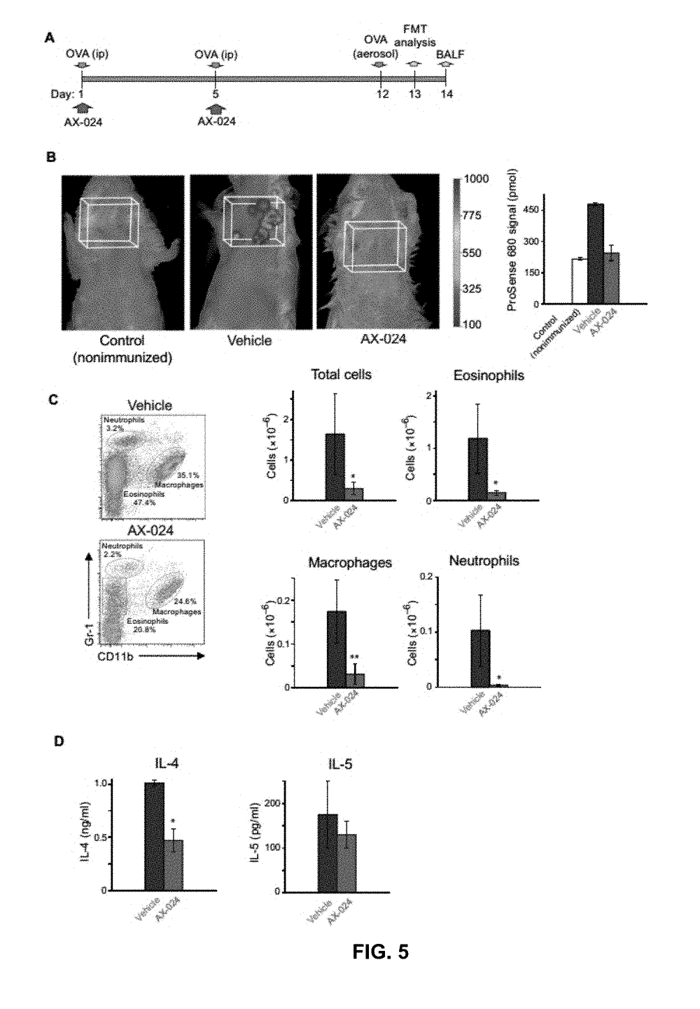

An animal model of ovalbumin (OVA)-induced allergic asthma was also used to test the prophylactic effect of AX-024. AX-024.HCl was administered twice, once with each OVA immunization (FIG. 5A). After challenge with an aerosol of OVA, inflammatory cell recruitment to the lungs of immunized mice was examined in live animals 24 hours later by quantitative fluorescence molecular tomography (FMT) (FIG. 5B) (35) and by postmortem evaluation of specific cell markers in the bronchoalveolar lavage fluid (BALF; FIG. 5C). Whereas nonimmunized mice had negligible numbers of airway inflammatory cells in BALF, the immunized vehicle group had considerable numbers of inflammatory eosinophils, macrophages, and neutrophils (FIG. 5C). Treatment with AX-024 significantly diminished the number of airway inflammatory cells in both assays. Allergic asthma is believed to be largely mediated by T_{H}2 cells (36), which are responsible for the secretion of IL-4, IL-5, and IL-13. Treatment with AX-024 significantly inhibited the release of IL-4 into BALF (FIG. 5D). These results suggest that AX-024 treatment also prevents sensitization of mice to inhaled antigens in a model of allergic airway inflammation.

### 2.5. Oral Administration of AX-024 Prevents the Development of Neurological Symptoms in a Model of Multiple Sclerosis

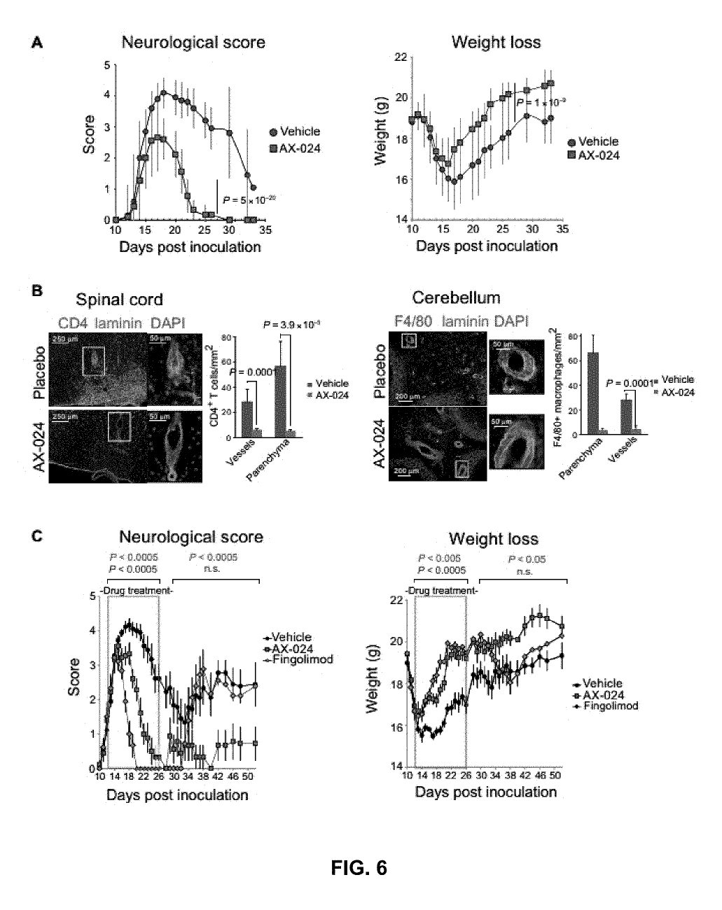

Multiple sclerosis (MS) is a chronic inflammatory demyelinating disease of the central nervous system (CNS) that is estimated to affect 2.3 million people worldwide (37). Experimental autoimmune encephalitis (EAE) is the most commonly used experimental model for MS, which is frequently induced by immunization with myelin-derived antigens in adjuvant to reproduce key pathological features of MS such as inflammation, demyelination, axonal loss, and gliosis (38). To assess the capability of AX-024 to prevent EAE in C57BL/6 mice immunized with myelin oligodendrocyte glycoprotein (MOG35-55), we administered a single daily dose of AX-024.HCl (10 mg/kg) by oral gavage for 10 days, starting at the date of immunization. Treatment with AX-024 significantly diminished the neurological symptoms and weight loss induced by MOG immunization (FIG. 6A). Mice receiving AX-024 rapidly recovered from neurological impairment and weight loss, becoming symptom-free by day 30, unlike mice that received the vehicle, in which ataxia and loss of the righting reflex persisted (FIG. 6A). All mice in both groups were sacrificed at day 33 and analyzed for CNS infiltration. A massive infiltration of blood vessels and neural parenchyma by CD4+ T cells and macrophages was detected in the cerebellum and spinal cord of the vehicle group, but not of the AX-024-treated group (FIG. 6B).

The effect of AX-024 was compared with that of an immunomodulatory drug currently used for MS treatment (glatiramer acetate) and with the immunosuppressor drug cladribine (39), all administered in single daily doses for 10 days, starting on the day of MOG immunization. AX-024 was more effective than both control drugs in this prophylactic setting at preventing weight loss and neurological symptoms (FIG. S10).

### 2.6. AX-024 Exerts a Long-Lasting Therapeutic Effect in the EAE Model of MS

To assess whether AX-024 not only prevented EAE symptoms but also had a therapeutic effect in this MS model, we treated MOG-immunized mice with single daily doses of AX-024.HCl (10 mg/kg, oral administration) starting after disease onset on day 13, once the symptoms of neurological impairment (clinical score, ≥2) and weight loss were already evident (FIG. 6C). A third group of mice was treated orally with fingolimod (0.6 mg/kg), a sphingosine 1-phosphate receptor (S1PR) modulator that has been approved as a first-line oral agent for the treatment of relapsing-remitting MS (40). Both treated groups rapidly recovered from neurological symptoms and gained weight, although the fingolimod group reached a score of 0 one week earlier than the AX-024 group (FIG. 6C). At day 26, drug treatment to both groups was discontinued, whereas scoring and weight were still monitored. By day 33, neurological symptoms and weight loss began worsening in the fingolimod group, reaching the values of the vehicle group by day 35. This outbreak of symptoms did not occur in the AX-024-treated mice, which maintained low clinical scores and kept gaining weight even up to day 52, 26 days after discontinuing the treatment (FIG. 6C). These results suggest that AX-024 exerts a therapeutic effect that lasts even after the drug is no longer present.

### 2.7. AX-024 Inhibits Effector TH Cell Differentiation Toward Proinflammatory Subsets

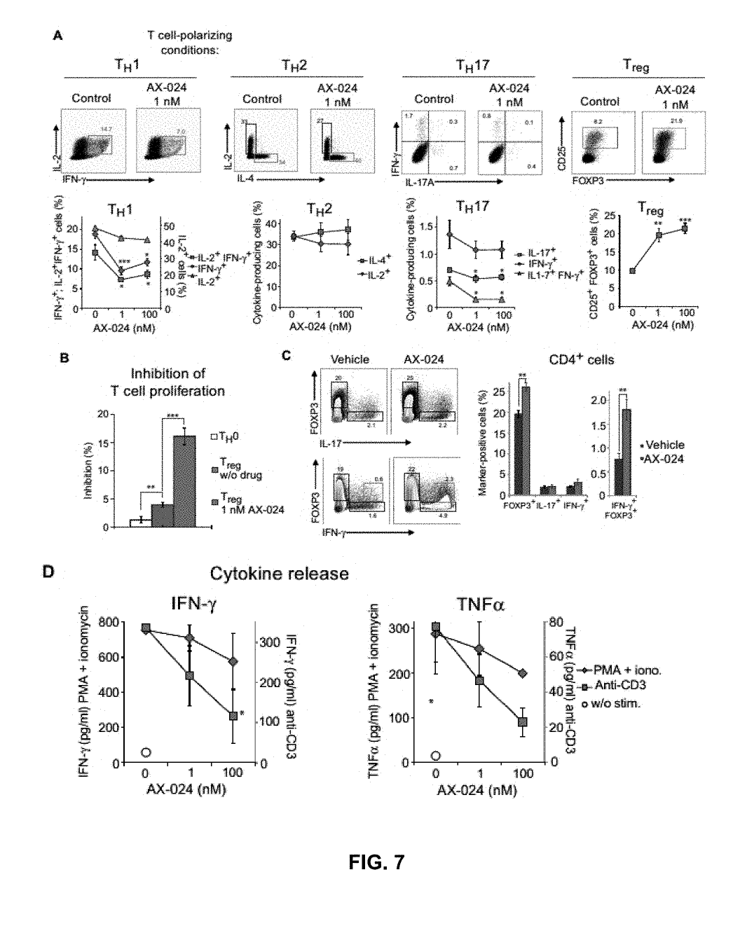

One possible mechanism by which AX-024 could exert its long-lasting effects is by altering T cell differentiation. Differentiation of naïve CD4⁺ T cells toward effector T cells not only depends on the presence of polarizing cytokines and their receptors but also on the signal strength of the TCR (41). Because AX-024 inhibited but did not block the earliest TCR signals (FIG. 3C), we could hypothesize that it altered the quality of effector cell differentiation. We found that 1 nM AX-024 inhibited the differentiation of human CD4+ T cells toward IFN-γ-, IL-2-, and IL17A-producing cells under T_{H}1-, T_{H}2-, and T_{H}17-polarizing conditions (FIG. 7A). However, AX-024 treatment increased differentiation toward IL-4-producing and FOXP3 (forkhead box P3)-expressing cells under T_{H}2-favoring and T_{reg}(regulatory T cell)-favoring conditions, respectively (FIG. 7A). The lack of inhibition or even potentiation toward T_{H}2 cells in these ex vivo experiments is incontrast with the observed reduction of IL-4 in BALFs in the asthma model (FIG. 5D), so we suspect that the discrepancy may be caused by the different milieu of the in vivo and ex vivo approaches. The pro-differentiation effect of AX-024 toward T_{reg} was evident not only through the expression of markers but also in a functional assay of naïve CD4⁺ T cell proliferation (FIG. 7B). To examine the effect of treatment with AX-024 on the differentiation of CD4+ T cells in vivo, we used the prophylactic EAE model to analyze the cytokine- and FOXP3-expressing populations in the draining lymph nodes of MOG-immunized mice. Before the onset of neurological symptoms, we detected a significant increase in the percentage of FOXP3⁻ CD4⁺ T cells but no significant differences in the percentage of total IFN-γ+ and IL-17³¹ CD4⁺ T cells (FIG. 7C). On the other hand, we detected a clear increase in the percentage of double-positive IFN-γ+ FOXP3+ cells, a population that has been described as the first line of T_{regs} that suppresses initial inflammatory responses (42). These results indicate that by altering the quality or strength of the TCR signal, AX-024 inhibits CD4+ T cell differentiation toward proinflammatory T cell populations while simultaneously favoring their differentiation toward anti-inflammatory effector subsets. CD4+ T cells differentiated under T_{H}1 conditions in the presence of AX-024 secreted less IFN-γ and less TNFα to the culture supernatant in response to TCR triggering than in response to PMA+ionomycin, suggesting that a previous exposure to AX-024 conditioned TCR signaling in a permanent manner (FIG. 7D).

2.8. AX-024 Allows Harnessing of an Efficient Memory T Cell Response Against a Mouse Pathogen

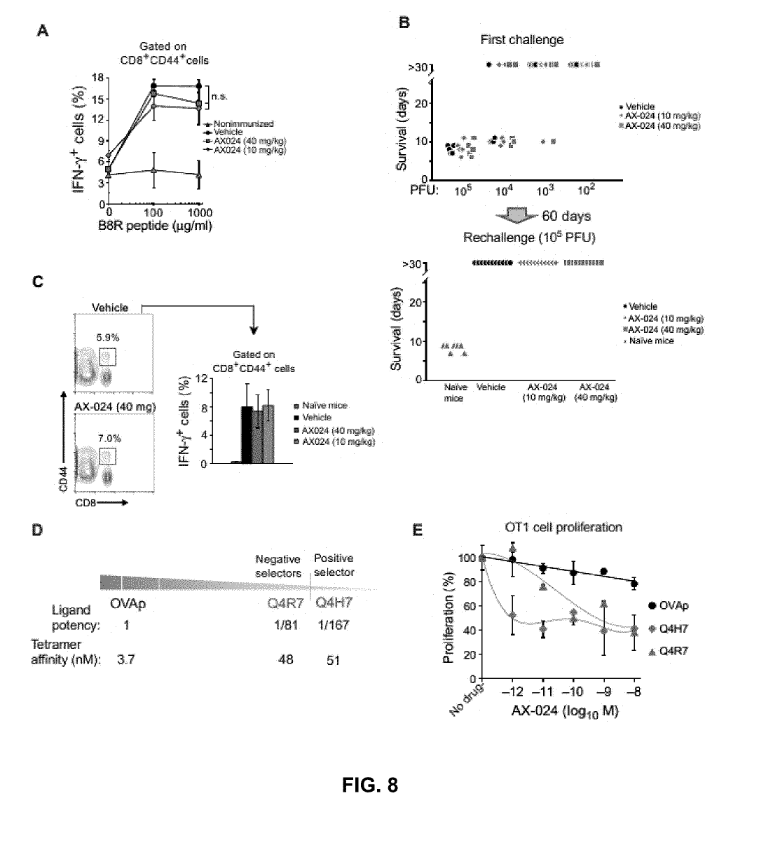

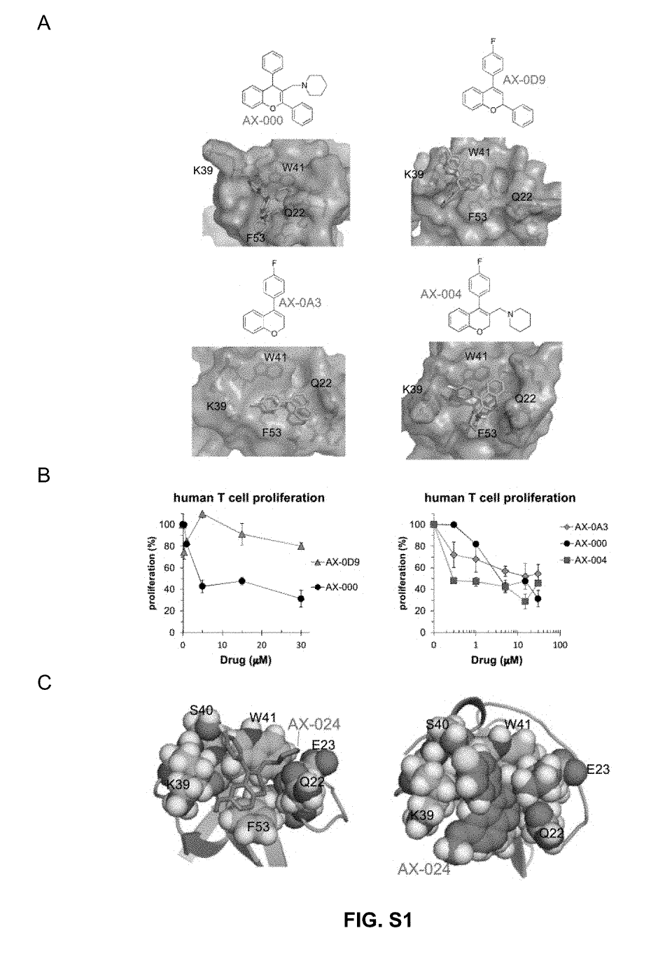

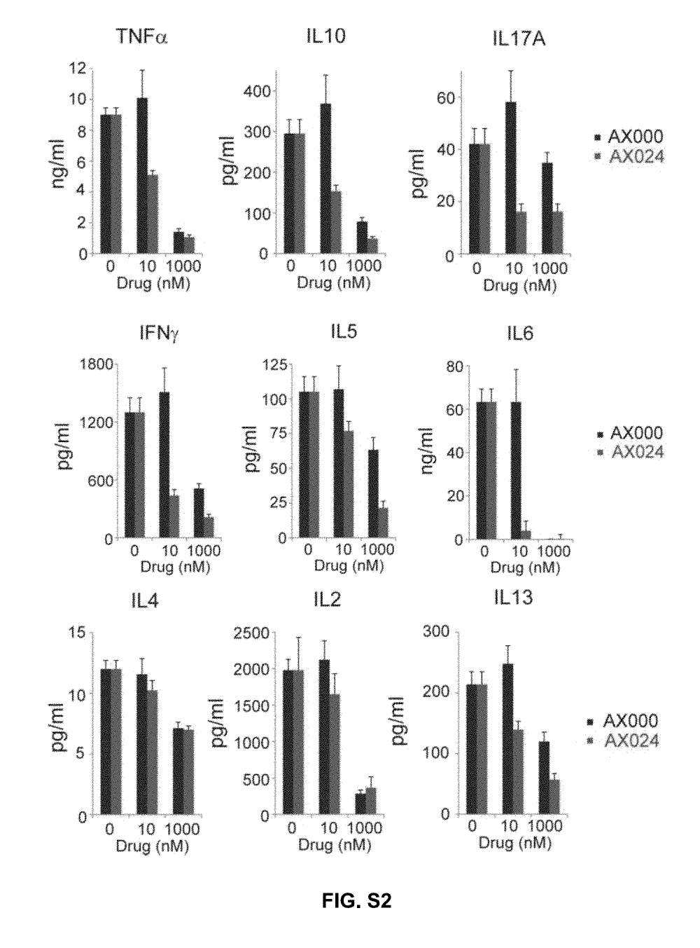

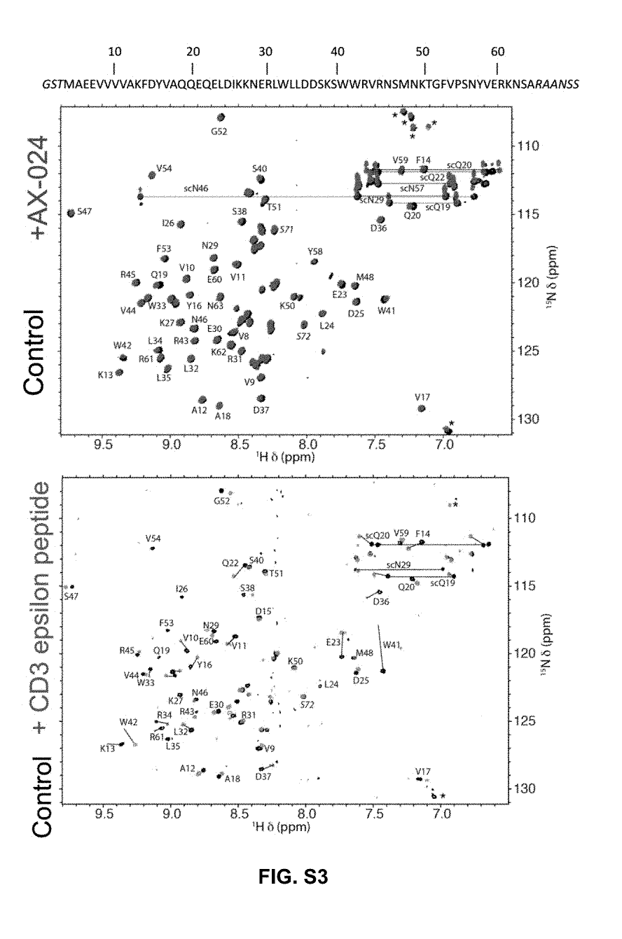

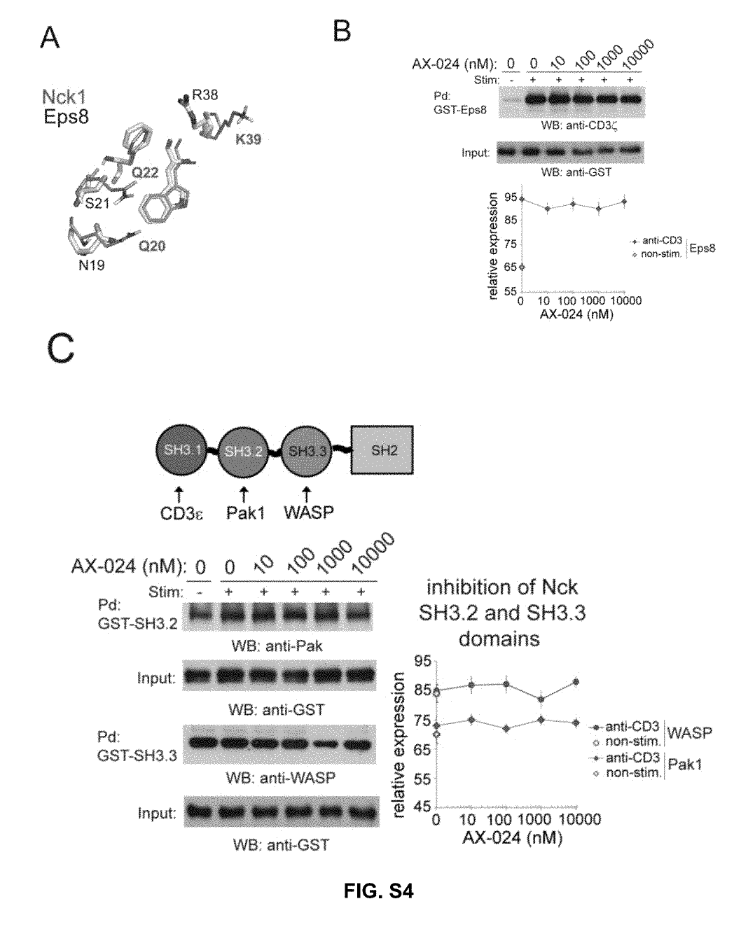

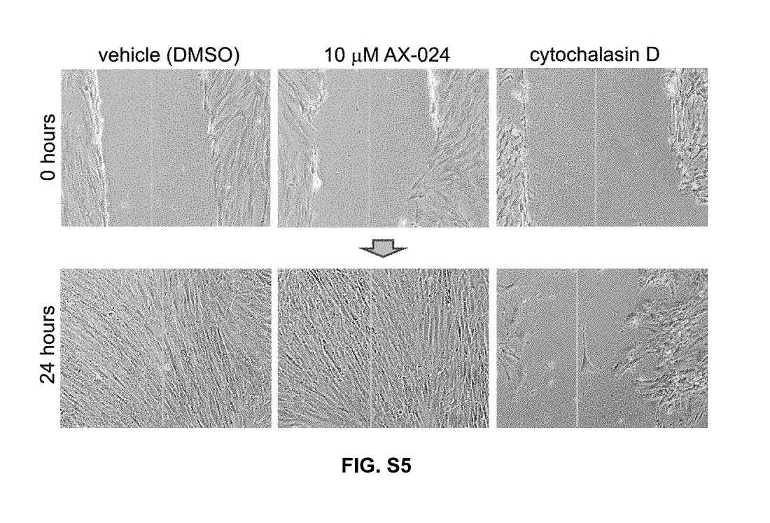

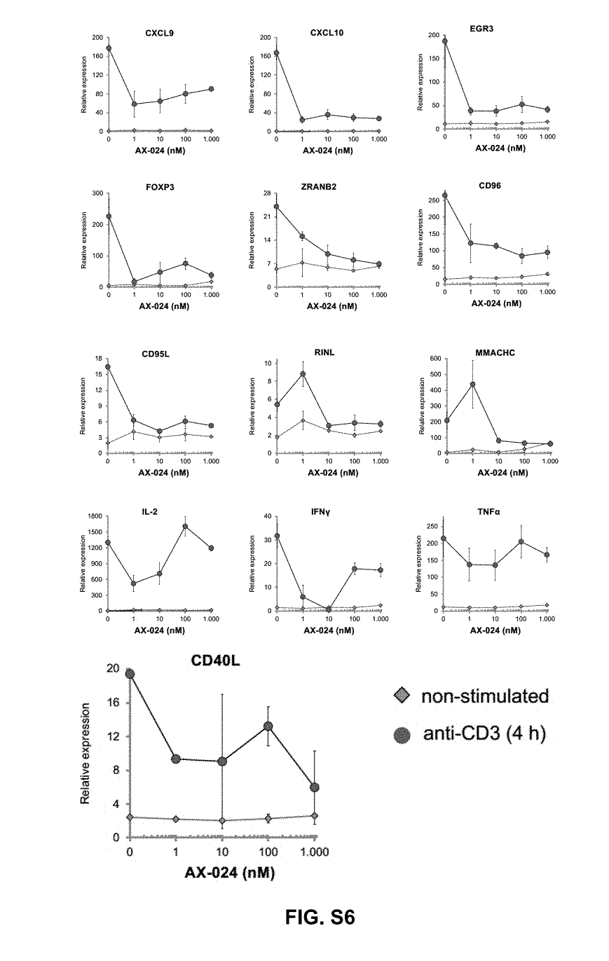

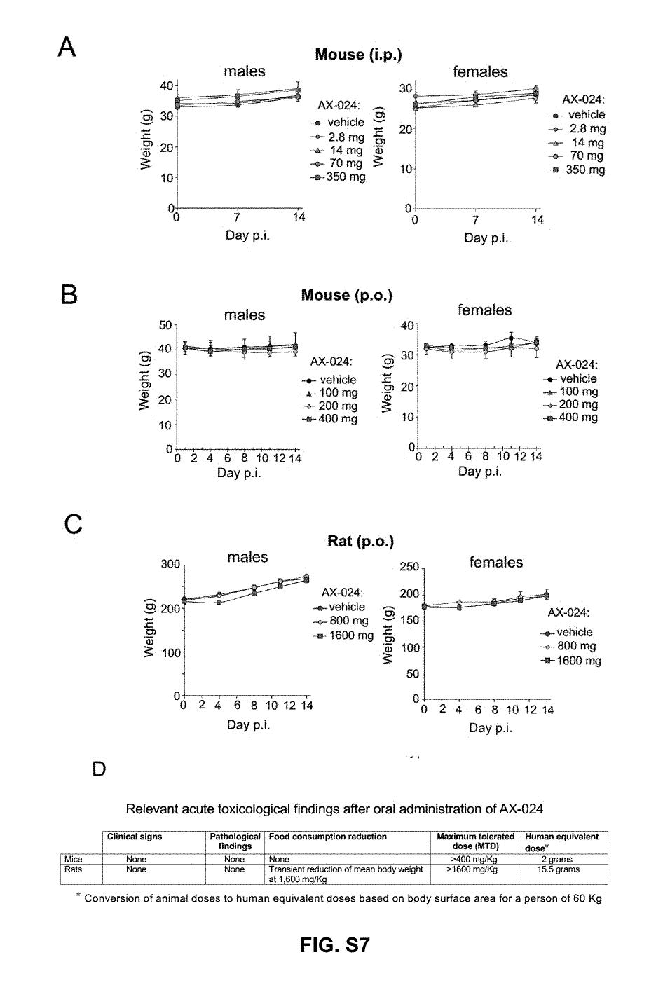

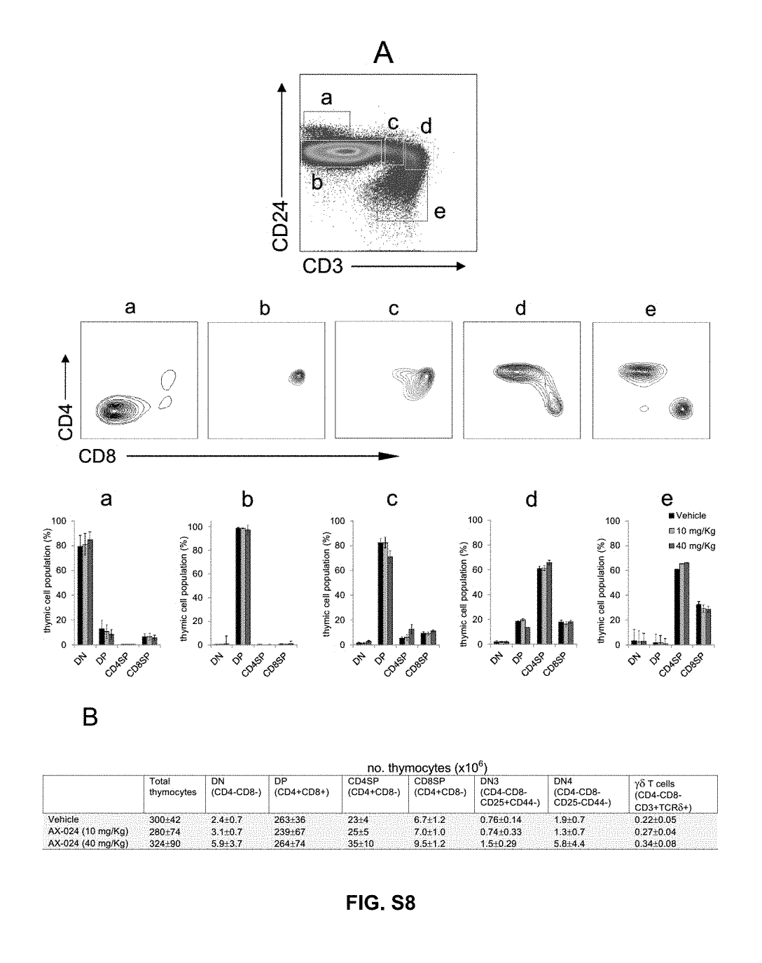

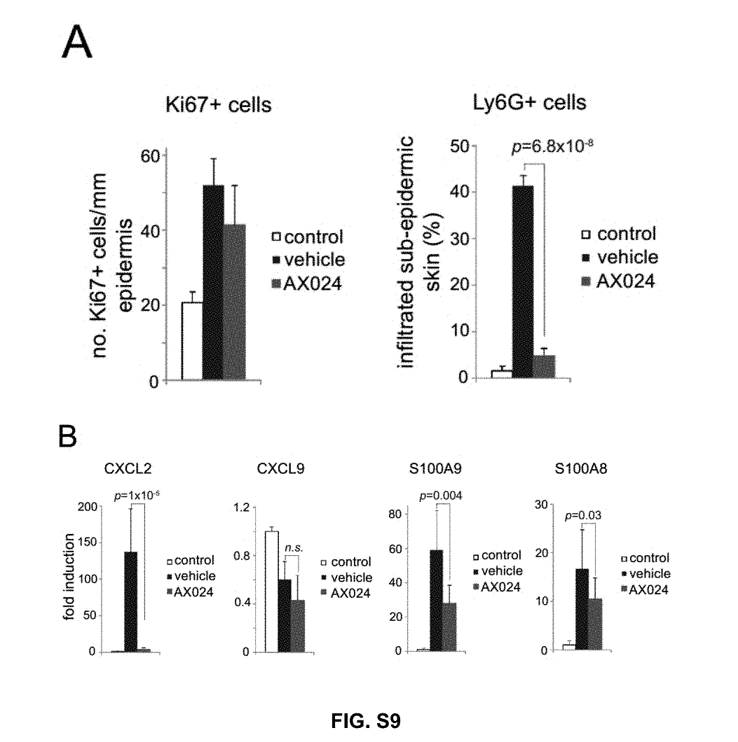

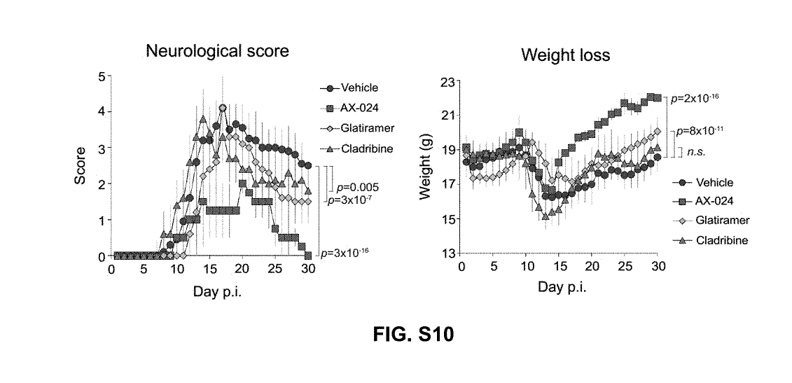

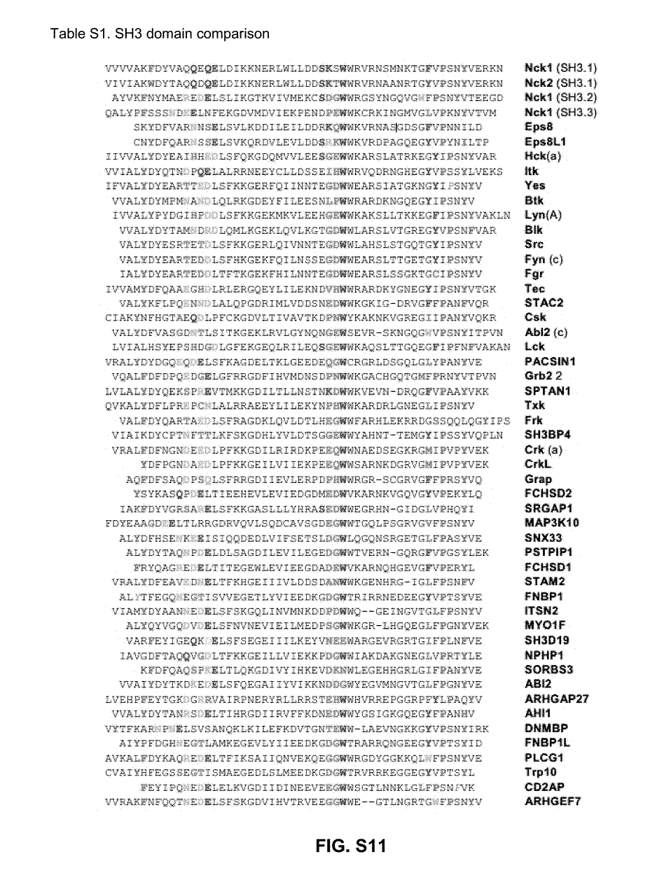

Considering that alteration of TCR signaling by AX-024 exerts a protective and therapeutic effect in the AD models described above, we wanted to assess whether this phenomenon was causing a concomitant, generalized immunosuppression. We immunized controls and mice treated with daily doses of AX-024.HCl (10 or 40 mg/kg) with the immunodominant poxvirus CD8 T cell epitope B8R and measured the antigen dependent CD8 T cell response 7 days later, on the basis of IFN-γ production. Neither dose of the drug significantly inhibited the activation of CD8 T cell response to the viral epitope (FIG. 8A). We next tested whether AX-024 increased the susceptibility of mice to infection by the mouse pathogen ectromelia virus (ECTV), a poxvirus that causes a smallpox-like lethal disease in mice (mousepox). Resistance to this infection requires full assembly of innate and adaptive immune responses (43). Drug-treated mice were given daily oral doses of AX-024 (10 and 40 mg/kg) that had demonstrated effectiveness in EAE and other models described herein for up to 10 days during and after infection with different doses of ECTV. All mice succumbed to a lethal dose of ECTV [105 plaque-forming units (PFU), intranasally (in)] but partly resisted infection with doses close to the median lethal dose (104 to 103 PFU, in; FIG. 8B, first challenge). AX-024 treatment did not influence survival rates to these sublethal doses nor to a lower dose (102 PFU, in), indicating that the drug did not increase sensitivity to infection in mice. To determine whether exposure to AX-024 during infection with a sublethal dose of ECTV altered the assembly of a functional memory response, we subjected all mice surviving the first challenge (FIG. 8B) to reinfection 60 days later with a lethal dose (105 PFU, in). All mice previously exposed to the virus resisted the lethal dose, regardless of their AX-024 treatment history, whereas a control group of naïve mice all succumbed to the infection (FIG. 8B, rechallenge).

A protective memory response against ECTV requires both CD4 help, for an efficient humoral response, and CD8 T cell-mediated cytotoxicity (44). To determine the presence of virus-responsive T cells, we analyzed the IFN-γ response of CD8 T cells taken from maxillary lymph nodes of mice surviving rechallenge upon ex vivo stimulation with B8R. The percentage of responding cells from AX-024-treated mice was indistinguishable from that of the vehicle group (FIG. 8C). These results indicate that AX-024 does not prevent mice from establishing an effective memory response against a viral pathogen, further suggesting that the compound is not immunosuppressive. The mechanism for AX-024 selectivity on self-antigens versus pathogen-derived antigens is not completely understood, although a possible hint for its selectivity may be the differential requirement for Nck recruitment to the TCR in response to weak versus strong antigens (17, 20). To interrogate a possible selectivity for weak antigens, we used the well-studied OT1 TCR transgenic system with its ample variety of antigen peptide derivatives (FIG. 8D) (45). As shown before in FIG. 2D, AX-024 inhibited OT1 T cell proliferation in response to the subthreshold weak agonist Q4H7 at picomolar concentrations, whereas much higher concentrations were required to inhibit T cell proliferation in response to the above-threshold agonist Q4R7 and the strong agonist OVAp (FIG. 8E).

### Discussion

Here, we describe the discovery of a new type of immunomodulatory therapy that targets signal 1 for T cell activation (TCR signaling), exerting a prominent prophylactic and therapeutic effect in different models of ADs, while preserving T cell activation in response to pathogen-derived antigens. We targeted the druggable pocket in the SH3.1 domain of Nck using computational modeling and small-molecular weight compound libraries to subsequently validate the therapeutic potential of the lead compound in three animal models of ADs. Provided that the animal models used are sufficiently relevant for human disease, the differential effect of AX-024 on autoimmune versus infectious diseases would be expected to make patients less prone to immunosuppression and to opportunistic infections in the future. Because AX-024 targets the TCR signal, this inhibitor has the potential to become a broad-spectrum therapy for ADs and other inflammatory diseases. However, the present study still falls short of clarifying the mechanisms for the distinction between self-antigens and pathogen-derived antigens and of explaining the long-lasting effect of AX024 in AD models once the drug is no longer present.

Although Nck is a ubiquitous protein, the AX-024 inhibitor targets a noncanonical DY pocket in the SH3.1 domain formed by a constellation of residues that is unique to this domain. AX-024 did not inhibit binding of Eps8 family of proteins—the other only known family of proteins bearing a SH3 domain with a DY pocket—and, furthermore, did not inhibit the binding of c-Cb1 to the SH3.1 domain of Nck (which does not involve the DY pocket). Further proof-of-target specificity is provided by the fact that incubation with AX-024 did not elicit additional inhibitory effects on the activation of T cells already deficient in the recruitment of Nck to the TCR. These features predict that AX-024 acts as a specific inhibitor of TCR-triggered T cell activation, an idea supported by the low acute toxicity of AX-024 in vivo and its potent inhibition of TCR-triggered T cell proliferation despite showing no inhibitory effect on T cell proliferation triggered by IL-2 or PMA+ionomycin. Hence, AX-024 does not apparently affect general cellular processes but rather acts as a selective inhibitor of TCR signaling. Target specificity is also suggested by the inhibition of TCR-triggered actin polymerization, an effect that is expected for an inhibitor that targets Nck recruitment and in line with the fundamental role of Nck as a scaffold for proteins involved in actin cytoskeleton remodeling (14, 15). The fact that AX-024 directly targets TCR-associated signaling machinery is also reflected by the inhibition of TCR-triggered ZAP70 phosphorylation, another direct effector of the TCR, and the partial inhibition of CD3ζ phosphorylation.

Because AX-024 inhibits Nck recruitment to the TCR, and given that TCR signals are central in the activation of the adaptive immune response, it would appear that AX-024 is an immunosuppressive agent rather than an immunomodulatory agent. However, Nck is required for the activation of T cells by weak but not strong antigens (17), which seems to be the case for self-reactive T cells involved in the generation of ADs (2, 46, 47). Similarly, PRS mutations in CD3ε affect T cell responses to weak but not strong antigenic peptides (20). This indicates that Nck recruitment to the TCR is critical for the activation of T cells bearing weakly reacting TCRs, such as those that have escaped negative selection in the thymus and are therefore weakly reactive against MHC loaded with self-antigens (2, 48). We show that AX-024 inhibits OT1 T cell activation much more strongly in response to low-affinity than to high-affinity antigen peptides. This may be the reason why AX-024 does not render mice more sensitive to infection by ECTV or prevents the assembly of an efficient T cell memory response against it, because the virus bears high-affinity antigens.

We tested the therapeutic value of AX-024 in mouse models of psoriasis, allergic asthma, and MS, displaying a protective effect in all of them. Single daily oral administrations have prophylactic effects in the two models tested (psoriasis and EAE). More remarkable outcomes arise from comparing oral AX-024 and oral fingolimod administrations in a therapeutic setting in the EAE model. Both compounds showed a clear therapeutic effect, completely reducing the neurological score to 0. However, unlike mice treated with fingolimod, mice treated with AX024 did not worsen after drug removal, indicating that AX-024 has a long-lasting therapeutic effect. From a mechanistic point of view, the comparison between the effects of fingolimod and AX-024 is especially intriguing. Fingolimod is believed to prevent T cell egress from the lymph nodes, and thereby CNS infiltration, by modulating the chemotactic receptor S1PR (40). This effect is dependent on the continuous presence of the drug, such that interruption of the treatment allows T cells to infiltrate the CNS, causing a rapid and severe relapse (49). By contrast, the therapeutic effect of AX-024 persisted after removing the drug, suggesting that exposure to AX-024 induces a persistent modification of autoimmune T cells.

One of the repercussions of TCR signal alteration seems to be the differential effect of AX-024 on the in vitro differentiation of CD4+ T cells toward proinflammatory, IFN-γ-producing, and IL-17A-producing effector cells, while favoring differentiation to Treg. These data suggest that, by inhibiting Nck recruitment to the TCR with AX024, it is possible to differentially affect intracellular signaling pathways rather than the overall signal intensity, leading to a differential effect on the capacity of T cells to differentiate into effector T cells. The hypothesis that conventional effector CD4⁺ T cells (T_{H}1, T_{H}2, and T_{H}17) can be inhibited without affecting Treg activity was recently proven in genetically modified mice bearing a germline mutation in ZAP70 that makes this kinase sensitive to a small-molecule inhibitor (50). Unfortunately, a highly specific, cell-permeable inhibitor of ZAP70 suitable for treatment against human disease has yet to be reported, which sets forth AX-024 as the first inhibitor of a direct TCR effector that could be used in clinical trials. Thus far, an Investigational Medicinal Product Dossier has been issued and approved for clinical tests in human volunteers, and phase Ia/Ib clinical trials have already been conducted (https://clinicaltrials.gov/ct2/show/NCT02243683?term=Artax&rank=2; https://clinicaltrials.gov/ct2/show/NCT02546635?term=Artax&rank=1).

We should highlight that AX-024 is not an inhibitor of an enzymatic activity but rather disturbs a protein-protein interaction. To the best of our knowledge, there is no other cell-permeable small-molecule inhibitor that acts by binding to an SH3 domain. Therefore, AX-024 represents a first-in-class inhibitor that modulates cell signaling by targeting SH3 domains and in turn is a first-in-class inhibitor of TCR signals. Finally, the low-acute toxicity profile of AX-024 and its high potency and selectivity, together with the fact that it targets TCR signals, make AX024 a candidate for evaluation as an oral drug in clinical trials of psoriasis, MS, and, presumably, many other Ads.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

### REFERENCES

1. M. M. A. Fernando, C. R. Stevens, E. C. Walsh, P. L. De Jager, P. Goyette, R. M. Plenge, T. J. Vyse, J. D. Rioux, Defining the role of the MHC in autoimmunity: A review and pooled analysis. PLOS Genet. 4, e1000024 (2008).
2. G. P. Morris, P. M. Allen, How the TCR balances sensitivity and specificity for the recognition of self and pathogens. Nat. Immunol. 13, 121-128 (2012).
3. R. I. Nurieva, X. Liu, C. Dong, Molecular mechanisms of T-cell tolerance. Immunol. Rev. 241, 133-144 (2011).
4. H. von Boehmer, F. Melchers, Checkpoints in lymphocyte development and autoimmune disease. Nat. Immunol. 11, 14-20 (2010).
5. Y. Y. Wan, R. A. Flavell, How diverse—CD4 effector T cells and their functions. J. Mol. Cell Biol. 1, 20-36 (2009).
6. M. D. Rosenblum, I. K. Gratz, J. S. Paw, A. K. Abbas, Treating human autoimmunity: Current practice and future prospects. Sci. Transl. Med. 4, 125sr1 (2012).
7. E. J. Fox, R. W. Rhoades, New treatments and treatment goals for patients with relapsing remitting multiple sclerosis. Curr. Opin. Neurol. 25, S11-S19 (2012).
8. O. Neuhaus, B. C. Kieseier, H.-P. Hartung, Immunosuppressive agents in multiple sclerosis. Neurotherapeutics 4, 654-660 (2007).
9. P. J. Bugelski, R. Achuthanandam, R. J. Capocasale, G. Treacy, E. Bouman-Thio, Monoclonal antibody-induced cytokine-release syndrome. Expert Rev. Clin. Immunol. 5, 499-521 (2009).
10. P. E. Love, S. M. Hayes, ITAM-mediated signaling by the T-cell antigen receptor. Cold Spring Harbor Perspect. Biol. 2, a002485 (2010).
11. I. de Aós, M. H. Metzger, M. Exley, C. E. Dahl, S. Misra, D. Zheng, L. Varticovski, C. Terhorst, J. Sancho, Tyrosine phosphorylation of the CD3-e subunit of the T cell antigen receptor mediates enhanced association with phosphatidylinositol 3-kinase in Jurkat T cells. J. Biol. Chem. 272, 25310-25318 (1997).
12. L. M. DeFord-Watts, J. A. Young, L. A. Pitcher, N. S. C. van Oers, The membrane-proximal portion of CD3 e associates with the serine/threonine kinase GRK2. J. Biol. Chem. 282, 16126-16134 (2007).
13. P. Delgado, B. Cubelos, E. Calleja, N. Martinez-Martin, A. Ciprés, I. Mérida, C. Bellas, X. R. Bustelo, B. Alarcón, Essential function for the GTPase TC21 in homeostatic antigen receptor signaling. Nat. Immunol. 10,880-888 (2009).
14. L. Buday, L. Wunderlich, P. Tamás, The Nck family of adapter proteins: Regulators of actin cytoskeleton. Cell. Signalling 14,723-731 (2002).
15. M. Lettau, J. Pieper, A. Gerneth, B. Lengl-Janβen, M. Voss, A. Linkermann, H. Schmidt, C. Gelhaus, M. Leippe, D. Kabelitz, O. Janssen, The adapter protein Nck: Role of individual SH3 and SH2 binding modules for protein interactions in T lymphocytes. Proc. Natl. Acad. Sci. U.S.A. 19,658-669 (2010).
16. E. Roy, D. Togbe, A. Holdorf, D. Trubetskoy, S. Nabti, G. Küblbeck, S. Schmitt, A. Kopp-Schneider, F. Leithäuser, P. Möller, F. Bladt, G. J. Hämmerling, B. Arnold, T. Pawson, A. Tafuri, Fine tuning of the threshold of T cell selection by the Nck adapters. J. Immunol. 185,7518-7526 (2011).
17. E. Roy, D. Togbe, A. D. Holdorf, D. Trubetskoy, S. Nabti, G. Küblbeck, A. Klevenz, A. Kopp-Schneider, F. Leithäuser, P. Möller, F. Bladt, G. Hämmerling, B. Arnold, T. Pawson, A. Tafuri, Nck adaptors are positive regulators of the size and sensitivity of the T-cell repertoire. Proc. Natl. Acad. Sci. U.S.A. 107,15529-15534 (2010).
18. D. Gil, W. W. A. Schamel, M. Montoya, F. Sánchez-Madrid, B. Alarcón, Recruitment of Nck by CD3ε reveals a ligand-induced conformational change essential for T cell receptor signaling and synapse formation. Cell 109, 901-912 (2002).
19. R. Blanco, A. Borroto, W. Schamel, P. Pereira, B. Alarcon, Conformational changes in the T cell receptor differentially determine T cell subset development in mice. Sci. Signaling 7, ral15 (2014).
20. P. Tailor, S. Tsai, A. Shameli, P. Serra, J. Wang, S. Robbins, M. Nagata, A. L. Szymczak-Workman, D. A. A. Vignali, P. Santamaria, The proline-rich sequence of CD3ε as an amplifier of low-avidity TCR signaling. J. Immunol. 181, 243-255 (2008).
21. O. Aitio, M. Hellman, T. Kesti, I. Kleino, O. Samuilova, K. Pääkkönen, H. Tossavainen, K. Saksela, P. Permi, Structural basis of PxxDY motif recognition in SH3 binding. J. Mol. Biol. 382, 167-178 (2008).
22. C. M. Santiveri, A. Borroto, L. Simón, M. Rico, B. Alarcón, M. A. Jiménez, Interaction between the N-terminal SH3 domain of Ncka and CD3ε-derived peptides: Non-canonical and canonical recognition motifs. Biochim. Biophys. Acta 1794, 110-117 (2009).
23. K. Takeuchi, H. Yang, E. Ng, S.-y. Park, Z.-Y. Sun, E. L. Reinherz, G. Wagner, Structural and functional evidence that Nck interaction with CD3ε regulates T-cell receptor activity. J. Mol. Biol. 380, 704-706 (2008).
24. S. S.-C. Li, Specificity and versatility of SH3 and other proline-recognition domains: Structural basis and implications for cellular signal transduction. Biochem. J. 390, 641-653 (2005).
25. A. M. Mongiovi, P. R. Romano, S. Panni, M. Mendoza, W. T. Wong, A. Musacchio, G. Cesareni, P. P. Di Fiore, A novel peptide-SH3 interaction. EMBO J. 18, 5300-5309 (1999).
26. Á. C. Cabrera, R. Gil-Redondo, A. Perona, F. Gago, A. Morreale, VSDMIP 1.5: An automated structure- and ligand-based virtual screening platform with a PyMOL graphical user interface. J. Comput. Aided Mol. Des. 25, 813-824 (2011).
27. A. Castello, M. Gaya, J. Tucholski, T. Oellerich, K.-H. Lu, A. Tafuri, T. Pawson, J. Wienands, M. Engelke, F. D. Batista, Nck-mediated recruitment of BCAP to the BCR regulates the PI(3) K-Akt pathway in B cells. Nat. Immunol. 14, 966-975 (2013).
28. A. Borroto, I. Arellano, R. Blanco, M. Fuentes, A. Orfao, E. P. Dopfer, M. Prouza, M. Suchànek, W. W. Schamel, B. Alarcón, Relevance of Nck-CD3ε interaction for T cell activation in vivo. J. Immunol. 192, 2042-2053 (2014).
29. A. Borroto, I. Arellano, E. P. Dopfer, M. Prouza, M. Suchànek, M. Fuentes, A. Orfao, W. W. Schamel, B. Alarcón, Nck recruitment to the TCR required for ZAP70 activation during thymic development. J. Immunol. 190, 1103-1112 (2013).
30. M. Carducci, L. Perfetto, L. Briganti, S. Paoluzi, S. Costa, J. Zerweck, M. Schutkowski, L. Castagnoli, G. Cesareni, The protein interaction network mediated by human SH3 domains. Biotechnol. Adv. 30, 4-15 (2012).
31. T. Kesti, A. Ruppelt, J.-H. Wang, M. Liss, R. Wagner, K. Taskén, K. Saksela, Reciprocal regulation of SH3 and SH2 domain binding via tyrosine phosphorylation of a common site in CD3ε. J. Immunol. 179, 878-885 (2007).
32. S. M. Schleicher, Psoriasis: Pathogenesis, assessment, and therapeutic update. Clin. Podiatr. Med. Surg. 33, 355-366 (2016).
33. B. Flutter, F. O. Nestle, TLRs to cytokines: Mechanistic insights from the imiquimod mouse model of psoriasis. Eur. J. Immunol. 43, 3138-3146 (2013).
34. L. van der Fits, S. Mourits, J. S. A. Voerman, M. Kant, L. Boon, J. D. Laman, F. Cornelissen, A.-M. Mus, E. Florencia, E. P. Prens, E. Lubberts, Imiquimod-induced psoriasis-like skin inflammation in mice is mediated via the IL-23/IL-17 axis. J. Immunol. 182, 5836-5845 (2009).
35. H. Korideck, J. D. Peterson, Noninvasive quantitative tomography of the therapeutic response to dexamethasone in ovalbumin-induced murine asthma. J. Pharmacol. Exp. Ther. 329, 882-889 (2009).
36. B. Bosnjak, B. Stelzmueller, K. J. Erb, M. M. Epstein, Treatment of allergic asthma: Modulation of Th2 cells and their responses. Respir. Res. 12, 114 (2011).
37. C. E. Markowitz, Multiple sclerosis update. Am. J. Manag. Care 19, s294-s300 (2013).
38. E. Mix, H. Meyer-Rienecker, H.-P. Hartung, U. K. Zettl, Animal models of multiple sclerosis—Potentials and limitations. Prog. Neurobiol. 92, 386-404 (2010).
39. B. C. Kieseier, D. R. Jeffery, Chemotherapeutics in the treatment of multiple sclerosis. Ther. Adv. Neurol. Disord. 3, 277-291 (2010).
40. C. S. Garris, V. A. Blaho, T. Hla, M. H. Han, Sphingosine-1-phosphate receptor 1 signalling in T cells: Trafficking and beyond. Immunology 142, 347-353 (2014).
41. N. J. Tubo, A. J. Pagán, J. J. Taylor, R. W. Nelson, J. L. Linehan, J. M. Ertelt, E. S. Huseby, S. S. Way, M. K. Jenkins, Single naive CD4+ T cells from a diverse repertoire produce different effector cell types during infection. Cell 153, 785-796 (2013).
42. V. Daniel, H. Wang, M. Sadeghi, G. Opelz, Interferon-g producing regulatory T cells as a diagnostic and therapeutic tool in organ transplantation. Int. Rev. Immunol. 33, 195-211 (2014).
43. V. Panchanathan, G. Chaudhri, G. Karupiah, Correlates of protective immunity in poxvirus infection: Where does antibody stand? Immunol. Cell Biol. 86, 80-86 (2008).
44. G. Chaudhri, V. Tahiliani, P. Eldi, G. Karupiah, Vaccine-induced protection against orthopoxvirus infection is mediated through the combined functions of CD4 T cell dependent antibody and CD8 T cell responses. J. Virol. 89,1889-1899 (2015).
45. M. A. Daniels, E. Teixeiro, J. Gill, B. Hausmann, D. Roubaty, K. Holmberg, G. Werlen, G. A. Holländer, N. R. J. Gascoigne, E. Palmer, Thymic selection threshold defined by compartmentalization of Ras/MAPK signalling. Nature 444,724-729 (2006).
46. A. M. Bulek, D. K. Cole, A. Skowera, G. Dolton, S. Gras, F. Madura, A. Fuller, J. J. Miles, E. Gostick, D. A. Price, J. W. Drijfhout, R. R. Knight, G. C. Huang, N. Lissin, P. E. Molloy, L. Wooldridge, B. K. Jakobsen, J. Rossjohn, M. Peakman, P. J. Rizkallah, A. K. Sewell, Structural basis for the killing of human b cells by CD8+ T cells in type 1 diabetes. Nat. Immunol. 13, 283-289 (2012)
47. S. Enouz, L. Carrié, D. Merkler, M. J. Bevan, D. Zehn, Autoreactive T cells bypass negative selection and respond to self-antigen stimulation during infection. J. Exp. Med. 209, 1769-1779 (2012).
48. S. Koehli, D. Naeher, V. Galati-Fournier, D. Zehn, E. Palmer, Optimal T-cell receptor affinity for inducing autoimmunity. Proc. Natl. Acad. Sci. U.S.A. 111, 17248-17253 (2014).
49. Y. Yoshida, T. Tsuji, T. Fujita, T. Kohno, Relapse of experimental autoimmune encephalomyelitis after discontinuation of FTY720 (fingolimod) treatment, but not after combination of FTY720 and pathogenic autoantigen. Biol. Pharm. Bull. 34, 933-936 (2011).
50. B. B. Au-Yeung, S. E. Levin, C. Zhang, L.-Y. Hsu, D. A. Cheng, N. Killeen, K. M. Shokat, A. Weiss, A genetically selective inhibitor demonstrates a function for the kinase Zap70 in regulatory T cells independent of its catalytic activity. Nat. Immunol. 11, 1085-1092 (2010).
51. D. Weininger, SMILES, a chemical language and information system. 1. Introduction to methodology and encoding rules. J. Chem. Inf. Comput. Sci. 28, 31-36 (1988).
52. R. Gil-Redondo, J. Estrada, A. Morreale, F. Herranz, J. Sancho, A. R. Ortiz, VSDMIP: Virtual screening data management on an integrated platform. J. Comput. Aided Mol. Des. 23, 171-184 (2009).

1. A method for selectively inhibiting TCR-Nck interaction and/or TCR-triggered T cell activation, and/or for specifically inhibiting the earlies TCR signaling events, and/or for inhibiting effector T_{H} cell differentiation toward proinflammatory subsets, in a patient comprising administering to the patient a compound of Formula A 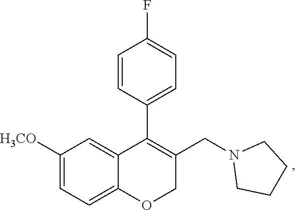 [SMILES: COc1ccc2c(c1)C(c1ccc(F)cc1)=C(CN1CCCC1)CO2]
or a pharmaceutically acceptable salt thereof.

2. The method of claim 1, wherein the compound selectively inhibits TCR-triggered T cell activation in the patient.

3. The method of claim 1, wherein the compound specifically inhibits the earlies TCR signaling events in the patient.

4. A method for treating a disease selected from psoriasis, asthma, and multiple sclerosis in a patient comprising administering to the patient a compound of Formula A, 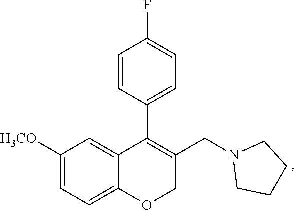 [SMILES: COc1ccc2c(c1)C(c1ccc(F)cc1)=C(CN1CCCC1)CO2]
or a pharmaceutically acceptable salt thereof.

5. The method of claim 4, wherein the disease is psoriasis.

6. The method of claim 4, wherein the disease is asthma.

7. The method of claim 6, wherein the compound attenuates severity of lung inflammation.

8. The method of claim 4, wherein the disease is multiple sclerosis.

9. The method of claim 8, wherein the compound attenuates severity of neurological symptoms in the patient.

10. The method of claim 1, wherein the compound inhibits effector T_{H} cell differentiation toward proinflammatory subsets in the patient.

11. A method for treating a T-cell mediated autoimmune and inflammatory disease, and/or for promoting T_{reg} differentiation, in a patient comprising administering to the patient a compound that inhibits an immediate TCR signal.

12. (canceled)

13. The method of claim 11, wherein the compound is a compound of Formula A 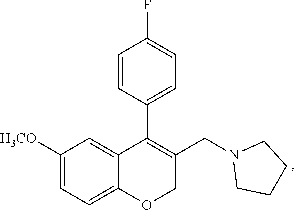 [SMILES: COc1ccc2c(c1)C(c1ccc(F)cc1)=C(CN1CCCC1)CO2]
or a pharmaceutically acceptable salt thereof.

14. The method of claim 4, wherein the compound preserves T cell activation in response to pathogen-derived antigens.

15. The method of claim 4, wherein the compound does not inhibit on T cell proliferation triggered by IL-2 or PMA+ ionomycin.

16. The method of claim 4, wherein the compound exerts a therapeutic effect that lasts after the drug is no longer detectable.

17. The method of claim 4, wherein the compound is administered orally.

18. The method of claim 5, wherein the compound attenuates severity of skin inflammation.

19. The method of claim 11, wherein the compound preserves T cell activation in response to pathogen-derived antigens.

20. The method of claim 11, wherein the compound does not inhibit on T cell proliferation triggered by IL-2 or PMA+ ionomycin.

21. The method of claim 11, wherein the compound exerts a therapeutic effect that lasts after the drug is no longer detectable.


SEQUENCE LISTING (71 sequences)

SEQ ID NO: 1 - protein, 8 aa, Artificial Sequence
  Description of Artificial Sequence Synthetic peptide
  MOD_RES (2)..(3): Any amino acid
  MOD_RES (5)..(6): Any amino acid
PXXPXXDY

SEQ ID NO: 2 - protein, 8 aa, Artificial Sequence
  Description of Artificial Sequence Synthetic peptide
SIIQFEHL

SEQ ID NO: 3 - protein, 8 aa, Gallus sp.
SIINFEKL

SEQ ID NO: 4 - protein, 8 aa, Artificial Sequence
  Description of Artificial Sequence Synthetic peptide
SIIQFERL

SEQ ID NO: 5 - protein, 17 aa, Homo sapiens
QRGQNKERPPPVPNPDY

SEQ ID NO: 6 - protein, 16 aa, Homo sapiens
RGQNKERPPPVPNPDY

SEQ ID NO: 7 - DNA, 19 bp, Artificial Sequence
  Description of Artificial Sequence Synthetic oligonucleotide
caggcggtgcctatgtctc

SEQ ID NO: 8 - DNA, 21 bp, Artificial Sequence
  Description of Artificial Sequence Synthetic oligonucleotide
gatcaccccgaagttcagtag

SEQ ID NO: 9 - DNA, 22 bp, Artificial Sequence
  Description of Artificial Sequence Synthetic oligonucleotide
gcaactgttcctgaactcaact

SEQ ID NO: 10 - DNA, 21 bp, Artificial Sequence
  Description of Artificial Sequence Synthetic oligonucleotide
atcttttggggtccgtcaact

SEQ ID NO: 11 - DNA, 19 bp, Artificial Sequence
  Description of Artificial Sequence Synthetic oligonucleotide
ccaaccaccaggctacagg

SEQ ID NO: 12 - DNA, 19 bp, Artificial Sequence
  Description of Artificial Sequence Synthetic oligonucleotide
gcgtcacactcaagctctg

SEQ ID NO: 13 - DNA, 21 bp, Artificial Sequence
  Description of Artificial Sequence Synthetic oligonucleotide
ggagttcgaggaaccctagtg

SEQ ID NO: 14 - DNA, 21 bp, Artificial Sequence
  Description of Artificial Sequence Synthetic oligonucleotide
gggatttgtagtggatcgtgc

SEQ ID NO: 15 - DNA, 21 bp, Artificial Sequence
  Description of Artificial Sequence Synthetic oligonucleotide
gcacagttggcaacctttatg

SEQ ID NO: 16 - DNA, 21 bp, Artificial Sequence
  Description of Artificial Sequence Synthetic oligonucleotide
tgattgtcctggtttgtgtcc

SEQ ID NO: 17 - DNA, 22 bp, Artificial Sequence
  Description of Artificial Sequence Synthetic oligonucleotide
aaatcaccatgccctctacaag

SEQ ID NO: 18 - DNA, 22 bp, Artificial Sequence
  Description of Artificial Sequence Synthetic oligonucleotide
cccacttttatcaccatcgcaa

SEQ ID NO: 19 - protein, 13 aa, Homo sapiens
RPPPVPNPDYEPI

SEQ ID NO: 20 - protein, 71 aa, Artificial Sequence
  Description of Artificial Sequence Synthetic polypeptide
GSTMAEEVVVVAKFDYVAQQEQELDIKKNERLWLLDDSKSWWRVRNSMNKTGFVPSNYVERKNSARAANSS

SEQ ID NO: 21 - protein, 56 aa, Homo sapiens
VVVVAKFDYVAQQEQELDIKKNERLWLLDDSKSWWRVRNSMNKTGFVPSNYVERKN

SEQ ID NO: 22 - protein, 56 aa, Homo sapiens
VIVIAKWDYTAQQDQELDIKKNERLWLLDDSKTWWRVRNAANRTGYVPSNYVERKN

SEQ ID NO: 23 - protein, 55 aa, Homo sapiens
AYVKFNYMAEREDELSLIKGTKVIVMEKCSDGWWRGSYNGQVGWFPSNYVTEEGD

SEQ ID NO: 24 - protein, 55 aa, Homo sapiens
QALYPFSSSNDEELNFEKGDVMDVIEKPENDPEWWKCRKINGMVGLVPKNYVTVM

SEQ ID NO: 25 - protein, 49 aa, Homo sapiens
SKYDFVARNNSELSVLKDDILEILDDRKQWWKVRNASGDSGFVPNNILD

SEQ ID NO: 26 - protein, 50 aa, Homo sapiens
CNYDFQARNSSELSVKQRDVLEVLDDSRKWWKVRDPAGQEGYVPYNILTP

SEQ ID NO: 27 - protein, 55 aa, Homo sapiens
IIVVALYDYEAIHHEDLSFQKGDQMVVLEESGEWWKARSLATRKEGYIPSNYVAR

SEQ ID NO: 28 - protein, 56 aa, Homo sapiens
VVIALYDYQTNDPQELALRRNEEYCLLDSSEIHWWRVQDRNGHEGYVPSSYLVEKS

SEQ ID NO: 29 - protein, 53 aa, Homo sapiens
IFVALYDYEARTTEDLSFKKGERFQIINNTEGDWWEARSIATGKNGYIPSNYV

SEQ ID NO: 30 - protein, 51 aa, Homo sapiens
VVALYDYMPMNANDLQLRKGDEYFILEESNLPWWRARDKNGQEGYIPSNYV

SEQ ID NO: 31 - protein, 56 aa, Homo sapiens
IVVALYPYDGIHPDDLSFKKGEKMKVLEEHGEWWKAKSLLTKKEGFIPSNYVAKLN

SEQ ID NO: 32 - protein, 53 aa, Homo sapiens
VVALYDYTAMNDRDLQMLKGEKLQVLKGTGDWWLARSLVTGREGYVPSNFVAR

SEQ ID NO: 33 - protein, 51 aa, Homo sapiens
VALYDYESRTETDLSFKKGERLQIVNNTEGDWWLAHSLSTGQTGYIPSNYV

SEQ ID NO: 34 - protein, 51 aa, Homo sapiens
VALYDYEARTEDDLSFHKGEKFQILNSSEGDWWEARSLTTGETGYIPSNYV

SEQ ID NO: 35 - protein, 51 aa, Homo sapiens
IALYDYEARTEDDLTFTKGEKFHILNNTEGDWWEARSLSSGKTGCIPSNYV

SEQ ID NO: 36 - protein, 55 aa, Homo sapiens
IVVAMYDFQAAEGHDLRLERGQEYLILEKNDVHWWRARDKYGNEGYIPSNYVTGK

SEQ ID NO: 37 - protein, 51 aa, Homo sapiens
VALYKFLPQENNDLALQPGDRIMLVDDSNEDWWKGKIGDRVGFFPANFVQR

SEQ ID NO: 38 - protein, 55 aa, Homo sapiens
CIAKYNFHGTAEQDLPFCKGDVLTIVAVTKDPNWYKAKNKVGREGIIPANYVQKR

SEQ ID NO: 39 - protein, 54 aa, Homo sapiens
VALYDFVASGDNTLSITKGEKLRVLGYNQNGEWSEVRSKNGQGWVPSNYITPVN

SEQ ID NO: 40 - protein, 56 aa, Homo sapiens
LVIALHSYEPSHDGDLGFEKGEQLRILEQSGEWWKAQSLTTGQEGFIPFNFVAKAN

SEQ ID NO: 41 - protein, 53 aa, Homo sapiens
VRALYDYDGQEQDELSFKAGDELTKLGEEDEQGWCRGRLDSGQLGLYPANYVE

SEQ ID NO: 42 - protein, 54 aa, Homo sapiens
VQALFDFDPQEDGELGFRRGDFIHVMDNSDPNWWKGACHGQTGMFPRNYVTPVN

SEQ ID NO: 43 - protein, 53 aa, Homo sapiens
LVLALYDYQEKSPREVTMKKGDILTLLNSTNKDWWKVEVNDRQGFVPAAYVKK

SEQ ID NO: 44 - protein, 52 aa, Homo sapiens
QVKALYDFLPREPCNLALRRAEEYLILEKYNPHWWKARDRLGNEGLIPSNYV

SEQ ID NO: 45 - protein, 55 aa, Homo sapiens
VALFDYQARTAEDLSFRAGDKLQVLDTLHEGWWFARHLEKRRDGSSQQLQGYIPS

SEQ ID NO: 46 - protein, 54 aa, Homo sapiens
VIAIKDYCPTNFTTLKFSKGDHLYVLDTSGGEWWYAHNTTEMGYIPSSYVQPLN

SEQ ID NO: 47 - protein, 53 aa, Homo sapiens
VRALFDFNGNDEEDLPFKKGDILRIRDKPEEQWWNAEDSEGKRGMIPVPYVEK

SEQ ID NO: 48 - protein, 49 aa, Homo sapiens
YDFPGNDAEDLPFKKGEILVIIEKPEEQWWSARNKDGRVGMIPVPYVEK

SEQ ID NO: 49 - protein, 49 aa, Homo sapiens
AQFDFSAQDPSQLSFRRGDIIEVLERPDPHWWRGRSCGRVGFFPRSYVQ

SEQ ID NO: 50 - protein, 49 aa, Homo sapiens
YSYKASQPDELTIEEHEVLEVIEDGDMEDWVKARNKVGQVGYVPEKYLQ

SEQ ID NO: 51 - protein, 49 aa, Homo sapiens
IAKFDYVGRSARELSFKKGASLLLYHRASEDWWEGRHNGIDGLVPHQYI

SEQ ID NO: 52 - protein, 52 aa, Homo sapiens
FDYEAAGDEELTLRRGDRVQVLSQDCAVSGDEGWWTGQLPSGRVGVFPSNYV

SEQ ID NO: 53 - protein, 51 aa, Homo sapiens
ALYDFHSENKEEISIQQDEDLVIFSETSLDGWLQGQNSRGETGLFPASYVE

SEQ ID NO: 54 - protein, 50 aa, Homo sapiens
ALYDYTAQNPDELDLSAGDILEVILEGEDGWWTVERNGQRGFVPGSYLEK

SEQ ID NO: 55 - protein, 48 aa, Homo sapiens
FRYQAGREDELTITEGEWLEVIEEGDADEWVKARNQHGEVGFVPERYL

SEQ ID NO: 56 - protein, 50 aa, Homo sapiens
VRALYDFEAVEDNELTFKHGEIIIVLDDSDANWWKGENHRGIGLFPSNFV

SEQ ID NO: 57 - protein, 51 aa, Homo sapiens
ALYTFEGQNEGTISVVEGETLYVIEEDKGDGWTRIRRNEDEEGYVPTSYVE

SEQ ID NO: 58 - protein, 50 aa, Homo sapiens
VIAMYDYAANNEDELSFSKGQLINVMNKDDPDWWQGEINGVTGLFPSNYV

SEQ ID NO: 59 - protein, 50 aa, Homo sapiens
ALYQYVGQDVDELSFNVNEVIEILMEDPSGWWKGRLHGQEGLFPGNYVEK

SEQ ID NO: 60 - protein, 50 aa, Homo sapiens
VARFEYIGEQKDELSFSEGEIIILKEYVNEEWARGEVRGRTGIFPLNFVE

SEQ ID NO: 61 - protein, 51 aa, Homo sapiens
IAVGDFTAQQVGDLTFKKGEILLVIEKKPDGWWIAKDAKGNEGLVPRTYLE

SEQ ID NO: 62 - protein, 48 aa, Homo sapiens
KFDFQAQSPKELTLQKGDIVYIHKEVDKNWLEGEHHGRLGIFPANYVE

SEQ ID NO: 63 - protein, 51 aa, Homo sapiens
VVAIYDYTKDKEDELSFQEGAIIYVIKKNDDGWYEGVMNGVTGLFPGNYVE

SEQ ID NO: 64 - protein, 53 aa, Homo sapiens
LVEHPFEYTGKDGRRVAIRPNERYRLLRRSTEHWWHVRREPGGRPFYLPAQYV

SEQ ID NO: 65 - protein, 51 aa, Homo sapiens
VVALYDYTANRSDELTIHRGDIIRVFFKDNEDWWYGSIGKGQEGYFPANHV

SEQ ID NO: 66 - protein, 53 aa, Homo sapiens
VYTFKARNPNELSVSANQKLKILEFKDVTGNTEWWLAEVNGKKGYVPSNYIRK

SEQ ID NO: 67 - protein, 51 aa, Homo sapiens
AIYPFDGHNEGTLAMKEGEVLYIIEEDKGDGWTRARRQNGEEGYVPTSYID

SEQ ID NO: 68 - protein, 53 aa, Homo sapiens
AVKALFDYKAQREDELTFIKSAIIQNVEKQEGGWWRGDYGGKKQLWFPSNYVE

SEQ ID NO: 69 - protein, 52 aa, Homo sapiens
CVAIYHFEGSSEGTISMAEGEDLSLMEEDKGDGWTRVRRKEGGEGYVPTSYL

SEQ ID NO: 70 - protein, 47 aa, Homo sapiens
FEYIPQNEDELELKVGDIIDINEEVEEGWWSGTLNNKLGLFPSNFVK

SEQ ID NO: 71 - protein, 51 aa, Homo sapiens
VVRAKFNFQQTNEDELSFSKGDVIHVTRVEEGGWWEGTLNGRTGWFPSNYV